(19) United States
(12) Patent Application Publication
(10) Pub. No.: US 2026/0070943 A1
(43) Pub. Date: Mar. 12, 2026

(54) A METHOD FOR HARVESTING PRODUCTS FROM HIGH CELL DENSITY PERFUSION CELL CULTURES

(71) Applicant: AMGEN INC., Thousand Oaks, CA (US)

(72) Inventors: Xiaoyang Zhao, Moorpark, CA (US); Oliver Kaltenbrunner, Moorpark, CA (US); Gregory T. Frank, Thousand Oaks, CA (US)

(21) Appl. No.: 19/109,673

(22) PCT Filed: Sep. 15, 2023
(86) PCT No.: PCT/US2023/032823

§ 371 Date: Mar. 7, 2025

Related U.S. Application Data
(60) Provisional application No. 63/407,437, filed on Sep. 16, 2022.

(51) Int. Cl. C07K 1/34; A61K 38/00; C07K 1/16; C07K 1/18; C07K 1/20; C07K 1/30; C07K 1/36; C07K 16/00

(52) U.S. Cl. CPC C07K 1/34; A61K 38/00; C07K 1/165; C07K 1/18; C07K 1/20; C07K 1/30; C07K 1/36; C07K 16/00; C07K 2317/31

(57) ABSTRACT

The disclosure provides methods of purifying a target protein, e.g., an antibody, from a host cell such as a mammalian cell using purification protocols incorporating harvest recovery operations comprising a continuous solids discharge disc stack centrifugation step followed by flocculation and depth filtration steps. The protein harvest methods of the disclosure recover high yields of purified target protein from perfusion cultures having a packed cell volume of greater than or equal to 16%, using an unconventional yet effective process.

### FIELD

The disclosure generally relates to the field of biomolecular processing, and more particularly to the field of harvesting biomolecules from perfusion cultures.

### BACKGROUND

Modern medicine faces many challenges in its effort to address a wide variety of diseases and conditions afflicting humans and other animal species. The development of new and better treatments extends beyond the efforts to develop new and better pharmaceuticals, including macromolecular biological compounds or biologics, such as monoclonal antibodies (mAbs), antibody fragments, antibody-like modalities, multispecific antibodies, engineered proteins, biosimilars, and the like. In recent years, those efforts have borne fruit at a steady pace, with an ever-increasing group of therapeutically active biologics approved for use in humans and filling the unmet needs of the medical community. A natural adjunct to the effort to develop new therapeutics is the effort to improve manufacturing processes to improve the yield of intact, functional therapeutic biologics while maintaining product quality, reducing the cost of production, increasing efficiency and sustainability, and streamlining production at pilot-, and commercial-scale production levels.

Manufacture of therapeutic biologics is a multi-step process involving production, recovery, and purification/polishing operations. Biologics are typically produced by expression from host cells in a cell culture, typically a mammalian cell culture for expression of the desired recombinant protein from host cells. Cell culture is followed by a harvest or recovery operation in which the culture medium containing the recombinant protein is removed from the bioreactor and an initial clarification is performed to prepare for the separation and purification of the target therapeutic protein from other components of cell culture broth. Such components include intact cells, cell debris, process- and product-related impurities including nucleic acids, such as DNA, proteins, such as non-target host cell proteins and high and low molecular weight variants of the desired therapeutic protein, lipids, carbohydrates, viral particles and the like.

As cell culture practices improve and packed cell volumes, cell densities, titer, process- and product-related impurities increase, pressure is placed on the downstream processes to effectively separate and purify the target protein. This is particularly true for harvest operations for perfusion cultures, including continuous perfusion cultures, which can be overwhelmed by the increased packed cell volumes, increased impurity level and increased titers.

For the foregoing reasons, a need persists in the art for biomolecule (e.g., biologic) purification processes involving a harvest recovery step that efficiently and cost-effectively results in high yield separations of biologics from other cell culture components arising from turbid perfusion cultures with packed cell volumes (PCV) of at least 16% at the time of harvest.

### SUMMARY

The disclosure provides a method for separating a recombinant protein produced in a perfusion culture from at least one other perfusion culture component comprising: (a) harvesting a pool or eluate stream from a perfusion culture comprising the recombinant protein and at least one other perfusion culture component, the perfusion culture having a packed cell volume (PCV) of at least about 16% at harvest; (b) introducing the harvest pool or eluate stream into at least one continuous solids discharge disc stack centrifuge; (b) operating the continuous solids discharge disc stack centrifuge, thereby separating fluid components into a centrate and a high-density composition; (c) collecting the centrate; (d) adding a flocculant to the centrate at a temperature of 8° C. to 12° C.; and (e) subjecting the centrate to a filtration step.

In some embodiments, the perfusion culture of any of the disclosed methods is harvested from single-use bioreactor, such as a single-use 500 L bioreactor or a single-use bioreactor that is 2,000 L or more.

In some embodiments, the prefusion culture harvest pool or eluate stream of any of the disclosed methods had a turbidity of at least 180 NTU at harvest. In some embodiments, the prefusion culture harvest pool or eluate stream of any of the disclosed methods had a viable cell density of at least 2×10⁷ viable cells/ml at harvest or a viable cell density of at least 3×10⁷ viable cells/ml at harvest or a viable cell density of at least 5×10⁷ viable cells/ml at harvest.

In some embodiments, the prefusion culture of any of the disclosed methods had a PCV of at least about 20% at harvest, or at least about 24% at harvest, or at least about 26% at harvest, or at least about 30% at harvest.

In some embodiments, the temperature of the perfusion culture at harvest in any of the disclosed methods is about 8° C. to about 12° C. For example, the temperature of the perfusion culture at harvest in any of the disclosed methods is about 10° C.

In some embodiments, a continuous harvest eluate stream from the perfusion culture of any of the disclosed methods is introduced into the continuous solids discharge disc stack centrifuge.

In other embodiments, a discontinuous batch of perfusion harvest pool of any of the disclosed methods is introduced into the continuous solids discharge disc stack centrifuge.

In some embodiments, the turbidity of the of the centrate of any of the disclosed methods is greater than about 160 NTU, such as a turbidity greater than about 180 NTU, or greater than about 200 NTU, or greater than about 250 NTU, or greater than about 300 NTU, or greater than about 500 NTU, or greater than about 600 NTU, or greater than about 750 NTU.

In some embodiments, the flocculant is added to the centrate of any of the disclosed methods at a temperature of about 10° C. In an exemplary embodiment, the flocculant is poly(diallyldimethylammonium chloride) (pDADMAC) or its monomer diallyl dimethyl ammonium chloride (DADMAC), and pDADMAC or DADMAC is added to at least 0.04% (w/w), or is added to 0.04-0.15% (w/w), or is added to 0.05% (w/w).

In any of the disclosed methods, the recombinant protein is a eukaryotic protein, such as a mammalian protein. In some embodiments, the mammalian protein is an antigen-binding protein.

In any of the disclosed methods, the recombinant protein is a mammalian antigen-binding protein and the antigen-binding protein is a monoclonal antibody, a bispecific antibody, a multispecific antibody, a bispecific T-cell engager molecule (BiTE®).

In other embodiments, the protein of any of the disclosed methods is a granulocyte colony-stimulating factor, an erythropoiesis stimulating agent, a HER receptor, a cell adhesion molecule, a growth factor, an osteoinductive factor, insulin, a coagulation protein, a colony stimulating factor, a blood group antigen; a growth hormone, a growth hormone receptor, a T-cell receptor; a neurotrophic factor, a neurotrophin, a relaxin, an interferon, an interleukin, a viral antigen, a lipoprotein, an integrin, a rheumatoid factor, an immunotoxin, a surface-membrane protein, a transport protein, a homing receptor, an addressin, a regulatory protein, or an immunoadhesin.

In some embodiments, the filtration step in any of the disclosed methods includes a depth filtration. Exemplary depth filters that may be used in any of the disclosed methods include MILLISTAK+® C0HC filter, a MILLISTAK+® C0SP filter, a SARTOCLEAR® DL60 filter, or a SARTOCLEAR® DL75 filter. In one embodiment, the depth filter is MILLISTAK+® C0HC filter or a MILLISTAK+® C0SP filter and wherein the centrate passes through the depth filter at a flux rate of 150 LMH or less and at a pressure of 10 psi or less. In some embodiments, the pressure is 2 psi or less. In some embodiments, the flux rate is 90 to 150 LMH.

The disclosure also provides for any of the disclosed methods which further comprise at least one additional chromatography step. Exemplary additional chromatography steps include ion exchange chromatography, hydrophobic interaction chromatography, or multimodal chromatography.

In addition, the disclosure provides for any of the disclosed methods which further comprise at least one or more viral filtration step, viral inaction step, and/or UFDF step.

The disclosure provides for a target protein made by any of the methods disclosed herein.

In another embodiment, the disclosure provided provide a method for maintaining low differential pressure during depth filtration of a load feed derived from a perfusion culture, the method comprising the steps: obtaining a flocculated centrate derived from a perfusion culture having packed cell volume (PCV) of at least 16% at the time of harvest; passing the centrate through a depth filter at a flux rate of 150 LMH or less and a filter pressure differential pressure of 10 psi or less; and recovering the eluate.

The disclosure also provides for methods of producing an isolated, purified recombinant protein from a perfusion culture comprising the steps of (a) initiating a perfusion culture in a single use bioreactor; (b.) inoculating the bioreactor with cells engineered to recombinantly express a protein of interest; (c.) culturing the cells until the perfusion culture has a packed cell volume of at least 16%; (d.) reducing the temperature of the culture to between 8° C. and 12° C. and collecting the perfusion culture from the bioreactor as a harvest pool or eluate stream; (e.) introducing the harvest pool or eluate stream into at least one continuous solids discharge disc stack centrifuge, wherein the harvest pool or eluate stream is between 4° C. and 12° C.; (f.) collecting a centrate from the centrifuge; (g.) adding a flocculant to the centrate at a temperature of 8° C. and 12° C.; (h.) passing the flocculated centrate through a depth filter at a at a flux rate of 90 to 150 LMH and at a pressure of 10 psi or less, wherein the flocculated centrate is between 4° C. and 12° C.; (i.) subjecting the filtered centrate to one or more chromatography, filtration, and or UFDF unit operations; and (j.) obtaining an isolated, purified, recombinant protein.

In another embodiment, the disclosure provides for pharmaceutical compositions comprising the isolated, purified, recombinant protein produced with any of the methods disclosed herein.

### BRIEF DESCRIPTION OF THE DRAWINGS

FIG. 1. Effect of flocculation on small-scale normalized depth filter loading level. FIG. 1 shows normalized small-scale depth filtration performance using untreated and flocculated material from large-scale single use continuous solids discharge disc stack centrifugation. In all but one condition tested, centrate flocculation increased the normalized depth filter throughput relative to the control. Normalized throughput was calculated by dividing small-scale depth filtration final throughputs (L/m²) of the pDADMAC treated (dark grey) and untreated (light gray) conditions by the depth filtration final throughputs (L/m²) of the control conditions (black). Control conditions (black) were operated using untreated material paired with X0HC depth filters. pDADMAC conditions were operated using 0.05% (w/w; weight pDADMAC/weight centrate) pDADMAC treated centrate was paired with C0HC, C0SP, X0HC, X0SP, DL60, or DL75 depth filters. Untreated conditions were operated using untreated centrate with C0SP, C0HC or X0SP depth filters. The following runs were terminated early during small-scale depth filtration due to time constraints: xmAb Run 1, xmAb Run 2 and bispecific 2. Higher throughputs would have been achieved for these runs if the operating time was extended. Error bars represent ±1 standard deviation from the average value. Bsp=Bispecific.

FIG. 2. Effect of flocculation and small-scale and depth filtration on percent yield. FIG. 2 shows the small-scale flocculation and depth filtration step yields for experiments illustrated in FIG. 1. Flocculation followed by depth filtration conditions (dark grey) had similar or improved step yield when compared to the control condition (black) or untreated conditions (light grey). Percent yield was calculated by dividing measured total protein mass (g) in the depth filtrate pool by the measured total protein mass (g) in the centrate pool. pDADMAC conditions were operated using 0.05% w/w pDADMAC treated centrate paired with C0HC, C0SP, X0HC, X0SP, DL60, or DL75 depth filters. Untreated conditions were operated using untreated centrate with C0SP, C0HC or X0SP depth filters. Error bars represent ±1 standard deviation from the average value. Bsp=Bispecific.

FIG. 3. Effect of small-scale flocculation host cell protein (HCP) levels. FIG. 3 shows the small-scale flocculation and depth filtration HCP log reduction values (LRV) for experiments illustrated in FIG. 1. Flocculation followed by depth filtration conditions (dark grey) showed similar HCP clearance to the control condition (black) and the untreated conditions (light grey) after accounting for HCP assay variability. The HCP LRV were calculated by applying a logarithm base 10 transformation to the ratio of the measured HCP levels in the centrate to the the measured HCP levels in the depth filtrate pools. Control conditions (black) were operated using untreated material paired with X0HC depth filters. pDADMAC conditions were operated using 0.05% (w/w) pDADMAC treated centrate paired with C0HC, C0SP, X0HC, X0SP, DL60, or DL75 depth filters. Untreated conditions were operated using untreated centrate with C0SP, C0HC or X0SP depth filters. Bsp=Bispecific.

FIG. 4. Effect of small-scale flocculation and depth filtration DNA levels. FIG. 4 shows the small-scale flocculation and depth filtration DNA LRV for experiments illustrated in FIG. 1. Flocculation followed by depth filtration conditions (dark grey) showed similar or improved DNA clearance relative to the control condition (black) and the untreated conditions (light grey). The DNA LRV were calculated by applying a logarithm base 10 transformation to the ratio of the measured DNA levels in the centrate to the the measured DNA levels in the depth filtrate pools. Control conditions (black) were operated using untreated material paired with X0HC depth filters. pDADMAC conditions were operated using 0.05% (w/w) pDADMAC treated centrate paired with C0HC, C0SP, X0HC, X0SP, DL60, or DL75 depth filters. Untreated conditions were operated using untreated centrate with C0SP, C0HC or X0SP depth filters. Bsp=Bispecific.

FIG. 5. Effect of pDADMAC on centrate turbidity. FIG. 5 shows the effect centrifugation operating conditions and pDADMAC concentration on supernatant turbidity. Supernatant turbidity values were measured after incubation with pDADMAC and lab scale centrifugation. Variation in centrifuge feed flow rate and bowl speed were used to create variation in initial centrate turbidity. pDADMAC concentration greater than 0.04 (% w/w) reduced centrate turbidity for two model molecules tested, i.e., mAb 1 Run 2 (A) and xmAb Run 1 (B).

FIG. 6. Effect of pDADMAC concentration on small-scale depth filtration performance. FIG. 6 shows the effects of pDADMAC concentration on small-scale normalized depth filtration throughput using large-scale centrate from (A) mAb1 (Run 1) and (B) mAb 2. Increased depth filtration throughput was observed using 0.05% to 0.1% w/w pDADMAC for mAb1 and 0.035% to 0.1% w/w pDADMAC for mAb 2 when compared to the control (black). Untreated material was paired with X0HC depth filters. pDADMAC conditions were operated using 0.02%, 0.035%, 0.05% or 0.1% w/w pDADMAC treated centrate paired with C0HC depth filters. Normalized throughput was calculated by dividing small-scale depth filtration final throughputs (L/m²) of pDADMAC treated conditions by small-scale depth filtration final throughputs (L/m²) of the control untreated condition.

FIG. 7. Effects of pDADMAC and PEG3000 Treatment on Depth Filtration Performance. FIG. 7 shows a comparison of small-scale scale depth filtration performance between pDADMAC and pDADMAC+PEG3000 treated conditions using representative large-scale centrate from run mAb 1 Run 3. mAb1 Run 3 large-scale run was executed using a stainless-steel continuous discharge centrifuge. The initial untreated centrate turbidity value was similar to that observed using a single-use continuous solids discharge centrifuge and was deemed to be representative for depth filtration studies. Increased normalized depth filter throughput was observed for both pDADMAC and pDADMAC+PEG3000 treated centrate when compared to the control. Untreated material was paired with X0HC depth filter. pDADMAC at 0.05% w/w was paired with C0HC depth filter. pDADMAC (0.05% w/w)+PEG3000 was paired with C0HC and C0SP depth filters. Normalized throughput was calculated by dividing small-scale depth filtration final throughputs (L/m²) of pDADMAC and/or pDADMAC+PEG treated conditions by depth filtration final throughputs (L/m²) of the control untreated condition. Small-scale depth filtration was performed a day after the end of the process (Day 19) for the mAb1 Run 3 large-scale run. Normalized throughput was calculated by dividing small-scale depth filtration final throughputs (L/m²) of pDADMAC treated conditions by small-scale depth filtration final throughputs (L/m²) of the control untreated condition.

FIG. 8. Effect of flocculation on depth filtration continuous pressure trends. FIG. 8 shows the differential pressure profiles for large-scale depth filtration train performed using 0.05% w/w pDADMAC and C0HC filters (dark grey) in comparison to a control small-scale depth filtration experiment performed with untreated centrate and X0HC filters (black). Representative large scale runs used were: mAb1 Run 1, xmAb 1 Run 1, xmAb 1 Run 2 and bispecific 2, Run 1. Use of flocculation and C0HC depth filtration resulted in low and stable differential pressure profiles when compared to the X0HC control where a rapid increase in differential pressure was observed. Delta A pressure represents the differential pressure across the stage 1 depth filter. Delta B pressure represents the differential pressure across the stage 2 bioburden reduction filter. Note that data plotted by permeability trend in the y-axis showed no difference compared to the absolute pressure trends in this Figure.

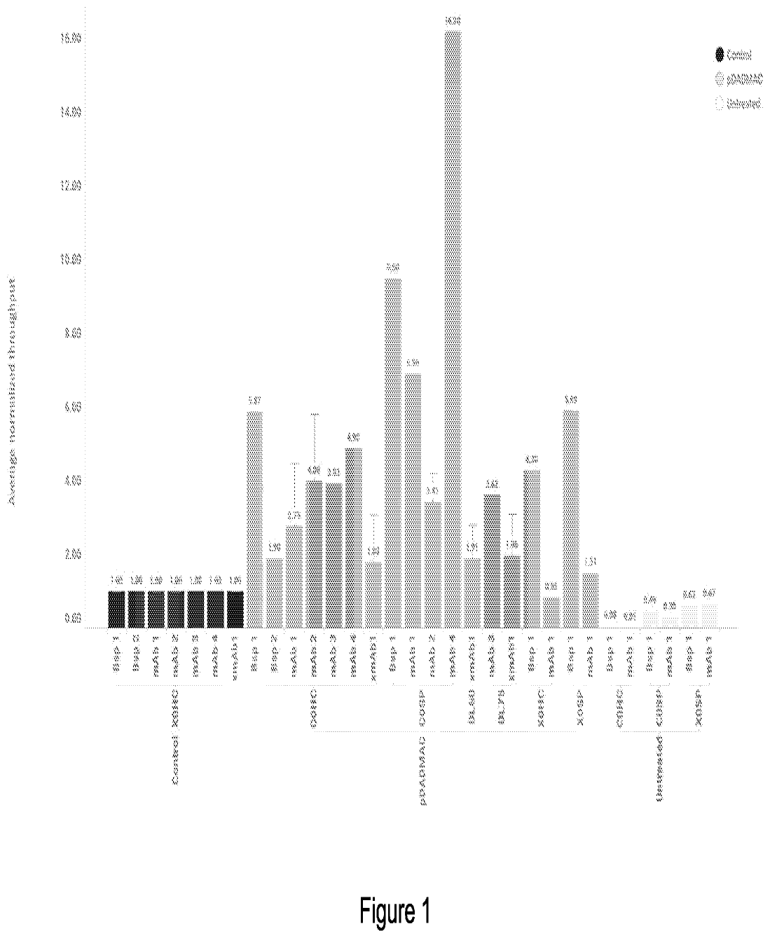

FIG. 1. Product quality comparison between pDADMAC treated material and untreated material. FIG. 9 shows a product quality comparison between pDADMAC-treated material and untreated material from xmAb1 Run 1. Flocculation followed by depth filtration conditions (light grey) showed similar product quality to the control condition (black) and the pDADMAC treated centrate (dark grey). Normalized product quality values were calculated by dividing the product quality values of the pDADMAC treated centrate (dark grey) and pDADMAC treated depth filtrate pool (light gray) conditions by product quality values of the untreated control conditions (black). Abbreviations: SE=size exclusion chromatography, CEX=cation exchange chromatography, rCE=reduced capillary electrophoresis with sodium dodecyl sulfate, nrCE=non reduced capillary electrophoresis; Main=median molecular weight range; Main peak=main or median peak resulting from chromatographic fractionation; LMW=low molecular weight; MMW=middle molecular weight.

### DETAILED DESCRIPTION

“Cell density” refers to the number of cells in a given volume of culture medium. “Viable cell density” (VCD) refers to the number of live cells in a given volume of culture medium, as determined by standard viability assays (such as the trypan blue dye exclusion method).

As used herein “packed cell volume” (PCV), also referred to as “percent packed cell volume” (% PCV), is the ratio of the volume occupied by the cells, to the total volume of cell culture, expressed as a percentage (see Stettler, et al., (2006) Biotechnol Bioeng. December 20:95(6): 1228-33). Changes in packed cell volume could arise from changes in cell diameter, Packed cell volume is a measure of the solid content in the cell culture. Solids are removed during harvest. More solids mean more effort to separate the solid material from the desired product during harvest. Also, the desired product can become trapped in the solids and lost during the harvest process, resulting in a decreased product yield. Since host cells vary in size and cell cultures also contain dead and dying cells and other cellular debris, packed cell volume is a more accurate way to describe the solid content within a cell culture than cell density or viable cell density. In addition, some cells, when in a growth-arrested state, will increase in size, so the packed cell volume prior to growth-arrest and post growth-arrest will likely be different, due to increase in biomass as a result of cell size increase.

“Centrate” as used herein is the low-density composition formed, or being formed, upon subjection of a cell culture broth to continuous solids discharge disc stack centrifugation, such as single-use continuous solids discharge disc stack centrifugation. In operation, the centrifuge fractionates a cell culture broth into the low-density composition and a high-density composition containing at least some of the solids originally found in the cell culture broth.

A “target protein” is any protein, peptide, or fragment thereof, existing in nature, produced synthetically, or expressed recombinantly, that is harvested according to the methods of the disclosure.

A “low-density composition” or “light phase” is a synonym for “centrate” that refers to the fluid exiting a disc stack centrifuge after removal of at least some solids from the input fluid.

A “high-density composition” or “heavy phase” is material enriched with particulate matter such as cells and cell debris resulting from the continuous solids discharge disc stack centrifugation.

A “bioreactor” is any container suitable for culturing cells capable of expressing a target protein. Bioreactors can range in size from laboratory scale to commercial scale and may have various enhancements including pump-controlled inlets and/or outlets, temperature gauges, CO₂ and or O₂ gauges, means for agitating a culture, and the like. A single use bioreactor is a bioreactor with a disposable bag instead of a culture vessel.

“Depth filtration” is filtration of a fluid comprising particles through a medium of sufficient depth to effect separation of particles of various sizes from the fluid.

“Tangential flow filtration” is filtration of a fluid comprising particles wherein the fluid is directed tangentially across the surface of the filtration medium, with transmembrane pressure, e.g., induced by gravity, resulting in some of the fluid entering the filtration medium to thereby be filtered.

“Q/Sigma” is the ratio of centrifuge feed flow rate to a theoretical equivalent settling area required to achieve an equivalent degree of solid-liquid separation with a settling pan. For disc stack centrifuge is calculated as:

$$\frac{π \cdot {ω}^{2}}{g} \cdot \frac{2}{3} \cdot N \cdot ( {{r}_{2}}^{2} - {{r}_{1}}^{2} ) \cdot cot α ,$$

where ω is the angular velocity; N is the number of disc; r₂ is the maximum radius of disc; r₁ is the minimum radius of disc; and α is the half-cone angle of disc.

“Load Factor” is the ratio of centrifuge feed flow rate to an empirical result-based separation area, KQ. KQ is calculated as:

$$280 \cdot {( \frac{n}{1000} )}^{1.5} \cdot N \cdot cot α \cdot ( {{r}_{2}}^{2.75} - {{r}_{1}}^{2.75} ) ,$$

where n is the bowl rotation speed in RPM; N is the number of disc; r₂ is the maximum radius of disc; r₁ is the minimum radius of disc; α is the half-cone angle of disc.

“Turbidity” is given its ordinary and accustomed meaning of the degree to which a fluid lacks clarity. As used herein, turbidity is measured in conventional Nephelometric Turbidity Units (NTUs) unless otherwise stated.

Samples from perfusion cultures with a PCV of at least 16% at the time of harvest were found to have higher turbidity levels. Turbidity is reduced during harvest, however, the greater the turbidity at harvest the more effort will be needed to separate the turbid matter from the desired product. The increased turbidity of the perfusion culture increases the risk for harvest equipment malfunction and/or decreased performance, resulting in a decreased product yield. Turbid perfusion cultures having a PCV of at least 16% are a challenge to traditional harvest operations. The turbidity was not completely reduced by centrifugation, turbidity values of as high as 1500 NTU were detected in samples of the light phase centrate collected from the single use continuous solids discharge disc stack centrifugation of perfusion cultures having a PCV of at least 16% at the time of harvest.

“Unit operation” refers to a functional step that is performed as part of the process of purifying a recombinant protein of interest. For example, a unit operation can include steps in operations such as, but not limited to, harvest, capture, purification, polish, viral inactivation, virus filtration, concentration and/or formulation the recombinant protein of interest. Unit operations can be designed to achieve a single objective or multiple objectives, such as a combination of capture and virus inactivation steps. Unit operations can also include holding or storing steps between processing steps.

“Perfusion culture” refers to a cell culture method wherein fresh media is fed into a bioreactor over the course of the cell culture, and the culture broth is passed through a cell retention device that selectively retains certain cell culture components (e.g., cells and optionally, recombinant proteins) in the bioreactor while removing spent media, byproducts, impurities and optionally, recombinant proteins in the permeate.

Fed-batch culture is the workhorse for producing therapeutic proteins. A fed-batch strategy is versatile and flexible and can be used at all bioreactor volumes. In a fed batch process, the culture is initiated at low cell densities at a fraction of the full working volume of the bioreactor, typically 50-75% volume and is supplemented with fresh nutrient media over the course of the culture, up to 100% of the full working volume of the bioreactor. The feed medium replenishes nutrients over the duration of the cell culture to prolong cell life and improve productivity. Spent media and waste products are not removed from the bioreactor. Cells, proteins, spent media, waste products, etc., are all retained in the bioreactor and accumulate over time. Fed batch cultures are therefore limited by the volume of the bioreactor, so larger bioreactors such as production bioreactors, operating at tens of thousands of liters, are favored to increase production. With time, fed batch culture will decline due to the buildup of spend media, waste products, etc., which negatively impact cell mass, productivity, and titer. Harvest typically begins when productivity declines. Typically, the packed cell volume (PCV) is less than or equal to 10% at the time of harvest.

In recent years, the use of perfusion has gained popularity for mammalian cell culture processes. Unlike fed-batch cultures, perfusion cultures are initiated at high cell densities at or near the full working volume of the bioreactor. Like fed-batch cultures, perfusion culture systems feed fresh media into a bioreactor over the course of the cell culture. Unlike fed-batch culture, a perfusion culture system makes use of pumps to direct the contents of the bioreactor through retention devices (filters, such as ultrafilters, microfilters, hollow fiber filters) that selectively retain or return certain cell culture components (cells and, optionally, recombinant proteins) back to the bioreactor. As such, perfusion culture can be maintained for a longer period than a fed-batch culture, for example, 15 days or more. Over the course of the perfusion culture a higher cell mass accumulates, which results in a larger quantity of cells, larger cell size, increased cell debris, byproducts and impurities at harvest compared to fed-batch cultures. Spent media (or nutrient depleted media), waste products, byproducts, product- and process-related impurities, recombinant proteins, and the like, are removed in the filter permeate as waste or collected for harvest. Perfusion cultures are harvested either through the retention devices and collected from the permeate, or bulk-harvested directly from the reactor. Perfusion processes are carried out in low-volume bioreactors (typically 500 L to 2,000 L) due to the limits of these retention devices. Such bioreactors include single use bioreactors.

Cell culture broth from perfusion cultures having a PCV of at least 16% at the time of harvest contain a high concentration of cell debris, product- and process-related impurities, byproducts, etc., which impacts protein passage through the filters and taxes the capacity of the pumping system, lowering harvest yields and increasing the level of impurities and byproducts in the harvest pool. PCV levels reaching 30% and above are not uncommon for perfusion cultures, in contrast to 10% PCV values often seen with fed-batch cultures. This high solid content increases the burden on downstream harvest, purification, and polish operations. Filters are more prone to plugging and clogging as the high solid content perfusion culture material is passed through, resulting in the need to replace filters during harvest, which takes time, reduces efficiency, lowers yield, and increases the cost of harvest. The pump capacity can also be limited by solution viscosity and high PCVs, which further impact the cost/efficiency of the harvest. The connection of the perfusion system and the retention device (tubing length, diameter, and the like) can compound the problem and increase the density of the culture broth entering the filters, resulting in cross-flow failure, lowering productivity in the bioreactor and negatively impacting harvest of the cell culture broth. The harvest capacity of perfusion cultures having a PCV of greater than 16% at the time of harvest typically reaches only about 70%, leaving a significant amount of the product behind.

Increased solid content in continuous perfusion cultures and the shortcomings of current retention systems used for perfusion culture have highlighted the need for improved perfusion culture harvest processes.

Harvest recovery processes for fed-batch cultures have traditionally relied on centrifugation, filtration, or a combination of these techniques to separate a target protein from culture particulates including cells, cell debris, and to a lesser extent, process- and product-related impurities, such as host cell proteins (HCP) and nucleic acids such as DNA. Intermittent solids discharge disc stack centrifugation is well-suited for clarifying these low solid content, fed-batch cultures and is widely used for fed-batch culture harvests. However, while intermittent disc stack centrifugation is suitable for clarifying low solid content, cell culture broths, there is a limit to its solid transportation capabilities. Moreover, as the cell culture solid content increases, the limitations of intermittent solids discharge disc stack centrifugation become apparent as the centrifuge bowl fills with cells and debris quicker, and therefore there is a need for more frequent solids discharge. Frequent solids discharge reduces product recovery yield and increases centrate turbidity, making downstream filtration more difficult. Intermittent solids discharge disc stack centrifugation is not a realistic viable option for high PCV perfusion cultures due to limited solid transportation capability’. Thus, intermittent solids discharge disc stack centrifugation is often coupled with another separation technique, such as filtration, or altering the size of impurity particles to accommodate the limitations of disc stack centrifugation, for example by creating larger particles through flocculation or precipitation of the cell culture broth prior to centrifugation.

Flocculated materials have larger average particle sizes than the native materials, resulting in more efficient clarification of fed-batch culture broths in a follow-on disc stack centrifugation run. This results in improved performance of disc stack centrifugation separation for fed-batch culture broth, and also facilitates more efficient clarification by depth filtration following such centrifugation, due to the reduced levels of small particulates that would otherwise contribute to the clogging of filter pores.

Filtration, in particular, depth filtration, has been used as both a primary harvest separation technique and as a partner to centrifugation, such as intermittent solids discharge disc stack centrifugation, to separate target biologics from fed-batch culture broths. Depth filters have become established as one of the main techniques routinely employed in fed-batch culture harvest recovery processes. Depth filters make use of a variety of porous filtration media, for example, diatomaceous earth, cellulosic, and/or synthetic fibers, creating a three-dimensional network of pores that collectively establish tortuous paths that materials must take to pass through the filter. Larger particulate matter from cell culture broths, e.g., cells and cell debris, can rapidly foul filters by plugging the pores or the surface of the filter.

For perfusion cultures, adoption of tangential flow microfiltration, in which the direction of cell culture broth flow is tangential to the direction of migration through the filter, is the primary means of harvest. During perfusion culture, as the cell culture broth passes through the filter, the larger particles (such as cells, and optionally recombinant protein) move across the surface of the filter and are returned to the bioreactor rather than settling into and clogging the pores of the filter. Further improvement has been achieved by the use of alternative tangential flow microfiltration in which the direction of cell culture broth across the surface of the membrane is periodically reversed, thereby providing a flushing action and improving the filter performance. The motivation to develop alternating tangential flow microfiltration highlights the fact that the particulate matter in perfusion culture broth tends to readily clog filters; however, the use of alternating tangential flow microfiltration for perfusion cultures having a PCV of at least 16% at harvest has become an ineffective solution to the problem of filter clogging, particularly when used for such perfusion cultures. The increasing solid content of perfusion cultures has resulted in the search for more effective harvest methods. Focus has been on improving filter technology, to increase the surface area of filters to accommodate the increased solids load. The results of this effort have not yet presented a solution for such cultures, and may not be able to solve the problem without increased costs for materials and labor and impacts to efficiency and productivity.

Use of perfusion culture as a component of commercial scale protein production, allows for scaled-down equipment size and facility footprint, reduced materials and environmental impact, incorporation of single use components, efficient and flexible production at all scales and with many therapeutic modalities. This provides strong incentives for developing more effective harvest processes that are capable of handling these higher solid content perfusion cultures. The disclosure herein relates to harvesting recombinant proteins produced by perfusion cultures having a PCV of at least 16% at harvest using a process previously not considered applicable to, or viable for, perfusion culture, i.e., centrifugation, specifically the use of a continuous solids discharge disc stack centrifuge. As described herein, centrifugation of perfusion cultures having a PCV of at least 16% results in an acceptable clearance of cellular material, however the centrate turbidity can be as high or higher than the harvest material and requires reduction before the centrate can be used with depth filtration.

Continuous perfusion cultures can achieve PCV of 16% to 45% or more in low culture volumes (2000 L or less) compared to a fed-batch culture which are less than 10% PCV in cell culture volumes 10-fold greater than perfusion cultures. This makes these high solid content perfusion cultures less suitable for filter harvest methods typically used with most perfusion culture harvests. The PCVs of these continuous perfusion cultures are also not comparable to fed-batch cultures, which have PCVs less than 10%, with higher culture volumes and higher yields that make them more suitable for larger scale intermittent or continuous solids discharge disc stack centrifugation and other centrifugation harvest methods. For commercial-scale perfusion cultures, harvest methodologies such as intermittent solids discharge centrifugation is not an option because at higher PCVs, the centrifuge bowl would be constantly open to eject solids, resulting in relatively ineffective separation of centrate from debris. Additionally, conventional stainless steel intermittent- or continuous-discharge centrifuges are not suitable for use in modern single-use manufacturing facilities (e.g., open floor plan design with minimal utilities) because the equipment does not have a closed processing design and requires clean-in-place and steam-in-place for product change over.

The introduction of continuous solids discharge disc stack centrifuge technology to the process is innovative in that the state of the art held the view that continuous solids discharge disc stack centrifugation could not discriminate between the densities of target proteins in cell culture broth and biological particulates such as cells, cell debris, host cell proteins (HCPs) or nucleic acids (e.g., DNA) in that fluid. Exemplary continuous solids discharge disc stack centrifuge system is a single-use continuous solids discharge disc stack centrifuge, and the single-use equipment has been expected to be too costly for long-term use. Intermittent solids discharge disc-stack centrifugation is disc stack centrifugation that generates cell paste continuously, accumulating the cell paste within the centrifuge bowl but ejecting the cell paste from the centrifuge periodically or intermittently. The solids ejection interval is based on cell culture PCV, cell culture harvest flow rate, and centrifuge bowl solids holding space. This type of centrifuge is typically used for lower density, lower PCV cell culture clarification. Continuous solids discharge disc stack centrifugation generates the cell paste continuously within the centrifuge bowl and transports the paste out of the centrifuge on a continuous basis at a pre-determined cell paste concentration. This type of centrifuge can be used for a much wider range of cell culture PCV than harvest methods based on intermittent solids discharge centrifugation. The development of disc stack centrifugation has provided a technique for separating substances on the basis of density differences that involves relatively low, or absent, shear forces, that is suitable for incorporation into harvest processes involving higher PCV perfusion cultures. Disclosed herein is the surprising finding that continuous solids discharge disc stack centrifugation effectively separates target proteins, such as antibodies, from biological particulates found in an actual perfusion culture broth having a PCV of at least 16%, particularly those with high harvest turbidities, with step yields of about 85% to 90% for target proteins, compared to about 70% step yields for target proteins using standard harvest technologies (e.g., microfiltration) for perfusion cultures. An exemplary system is a single-use continuous solids discharge disc stack centrifuge. The benefit of increased PCV with step yields of about 85% to 90%, coupled to the cost savings in reduced down-time and labor needed to unclog centrifuges, more than offsets the cost of single-use centrifugation, resulting in an economically attractive target protein harvest methodology. Incorporating continuous solids discharge disc stack centrifugation followed by clarification of the centrate using flocculation or precipitation provides a scalable means by which the protein may be harvested in a continuous stream over time, while still maintaining a clear flow path and minimizing fouling concerns for any subsequent filtration process steps, even with the higher solid content perfusion cultures.

Disc stack continuous solids discharge centrifugation was selected because it effectively removes intact cells and larger cell debris fragments without expanding the harvest volume, as with the typical microfiltration harvest, and can be operated using an open floor plan manufacturing facility due to its closed equipment design. When used in combination with a perfusion culture process, the centrate is free of intact cells but still contains a high concentration of particles too small to be separated by centrifugation. Turbidity levels of 1500 NTU were observed from the centrifuge harvest of these higher PCV perfusion cultures. These small particles clog depth filters and sterile filters at low loading capacities such that large membrane areas are required for filtration. Flocculation is employed to aggregate the small particles into large particles. This increases the average particles size in solution and decrease the total particle concentration. When paired with depth filter pore sizes that effectively remove the flocculated particles (e.g. C0HC), the depth filter and sterile filter loading capacities are improved, reducing the total membrane area required to filter the centrate at large scale.

In addition to improved step yield, a decreased pool volume and more consistent harvest performance from one lot to another were observed. Smaller pool volumes with consistent product quantity improve the purification facility fit and help optimize a manufacturing plant production schedule, raw material usage and labor allocations. Moreover, incorporation of a disc stack continuous discharge centrifugation step decreases the subsequent depth filtration loading level to a point where the depth filtration media has sufficient solids holding space to be effective in further purifying the target protein without immediate and unproductive clogging.

Through the use of continuous solids discharge disc stack centrifuge technology, cell culture broth is separated into heavy phase (solids) and light phase (centrate) streams. The methods of the disclosure described herein are applicable to several modes of centrifuge operation, specifically: (1) Continuous separation of protein from cellular material in a harvest stream of a higher solid content perfusion culture manufacturing process; (2) Continuous separation of protein and impurities from cellular material, followed by re-introduction of the cellular material back to the bioreactor; and (3) Effective batch harvest of product from a bioreactor, such as the continuous solids discharge disc stack centrifugation of a higher solid content perfusion culture where output from the bioreactor is held in a surge or hold tank for periodic delivery to the centrifuge.

In addition, the continuous solids discharge disc stack centrifuge presents a closed system that is scalable and can be used in the production of therapeutic target proteins without need for isolation rooms and other requirements to avoid contamination and ensure product quality. This method is compatible with the bioreactors favored for perfusion culture and open ballroom settings that have been adopted by many who use continuous perfusion culture in bioreactors. For example, single-use continuous solids discharge disc stack centrifuges are compatible with single-use bioreactors. Commercial scale, stainless steel intermittent solids discharge or continuous solids discharge disc stack centrifuges, even if they were usable, are not compatible with commercial scale single-use bioreactors and perfusion systems.

Suitable harvest stream flow rates from cell culture bioreactors depend on several process design decisions. In order to size the continuous solids discharge disc stack centrifuge equipment to fit the desired harvest flow rate, the operation of the unit may be either continuous, or operated in a periodic (batch) manner, as noted above. Typically, flow rates are adjusted to maintain a near-constant loading or Q/Sigma value.

In the methods described herein, the harvest operation begins with collecting the cell culture fluid directly from the bioreactor or from a surge or hold tank and subjecting it to centrifugation using a continuous solids discharge disc stack centrifuge. The centrate may also be collected into a harvest hold vessel or connected in-line to a surge vessel prior flocculation and loading onto one or more depth filters.

The continuous solids discharge disc stack centrifuge equipment itself may be constructed of stainless steel, plastic, or another material. When the equipment is a single-use continuous solids discharge disc stack centrifugation, it is combined with a single-use insert comprising the centrifuge bowl and other product-contacting surfaces or flow paths. The single-use inserts may be functionally closed with single-use aseptic connectors and sterilized by irradiation prior to use. The single-use insert aseptic connectors enable attachment to a bioreactor or surge tank as well as a centrate collection tank to functionally close the centrifugation operation.

The methods described herein subject culture broth to continuous solids discharge disc stack centrifugation in the first step of a harvest operation to recover the recombinant protein of interest from perfusion cultures having a PCV of at least 16%. In greater detail, it can be understood that the harvest operation of target protein (e.g., recombinant protein) has two parts: (1) the removal of cells, cell debris, and the like, and (2) clarification of the centrate containing the protein of interest. To achieve this result, the disclosed methods employ a continuous solids discharge disc stack centrifugation step. Any continuous solids discharge disc stack centrifuge known in the art is contemplated for use in the methods of the disclosure. As this system may be a single-use continuous solids discharge disc stack centrifugation, the components that contact the culture are single-use components that need not comprise materials and construction methods designed to withstand repeated use. Nonetheless, the single-use centrifuges contemplated for the disclosed methods may be used in continuous harvest methods of varying duration and, thus, the centrifuges may be built to remain operational for extended periods of use.

The continuous solids discharge disc stack centrifuge may be used to perform an initial separation of a culture broth into heavy phase (solids, including cells) and centrate (including secreted target protein (e.g., recombinant protein and solids that did not pass into the heavy phase)) phases before reaching a depth filtration step. The centrate is further clarified using flocculation prior to the depth filtration step. Continuous solids discharge disc stack centrifuges, including single use continuous solids discharge disc stack centrifuges, can be based on commercially available units from, e.g., Alfa Laval Corporate AB (Lund, Sweden), GEA Westfalia (Oelde, Germany). Continuous solids discharge disc stack centrifuges of all forms are suitable for use in a single-use format in accordance with the methods of the disclosure.

The continuous solids discharge disc stack centrifuge technology may be applied to the harvest of culture broth from a single use or stainless steel bioreactor. Preferably, single use bioreactors for use in such methods have a volume no greater than 5,000 L.

In some embodiments, centrifuge theoretical setting area (Sigma value) normalized flow rates, Q/Sigma, range disclosed herein as useful in recovering target proteins as 4.6E-9 to 2.2E-8 m/s. The harvest stream from the bioreactor depends on several process design decisions. As a result, there is a wide range around the harvest stream flow rates when compared across mammalian cell culture processes. For single-batch harvests, higher flow rates are preferred. For the continuous harvest strategies, such as cell-bleed product separation, lower flow rates are preferred. The continuous solids discharge disc stack centrifuge is typically operated in a continuous manner as cell culture batches are fed into it.

Successful continuous solids discharge centrifugation is characterized by high step yield and clearance of cells and cell debris (Table 1). For perfusion culture processes having a PCV of at least 16%, variability in suspended particle levels is observed. Therefore, the capability of the continuous solids discharge centrifuge to remove cells and cells debris was quantified by comparison of the light phase turbidity to an established baseline turbidity value. The baseline turbidity value was measured from cell culture supernatant generated after centrifugation of a 50 mL cell culture sample at approximately 2094 relative centrifugal force (rcf) for 17 minutes. Successful purification was achieved when approximately 100% of cells were removed from the light phase with high yield of the target protein, as disclosed herein. As shown in Table 1, effective cell and cell debris separation was observed; the light phase turbidity were less than 2-fold higher than the baseline turbidity values, however, this degree of turbidity is sufficient to impede subsequent purification by depth filtration. Centrate is generally maintained at 10° C., although the centrifuge may warm the light phase to 16-25° C. The centrate is held at 10° C. before and during flocculation. In experiments disclosed herein, the centrate was not sparged, but it is expected that centrates could be sparged, particularly if the centrate is stored for a period of time.

The level of sub-micron cellular debris, product- and process-related impurities, and other culture byproducts produced by the high solid content perfusion cultures described herein that were collected in the centrate were found to be significantly higher than that seen for fed-batch processes at any scale. The high degree of impurities in the centrate more easily and quickly foul depth and sterile filters, requiring additional filtration area to prepare the centrate for further processing. Therefore, further clarification of the high turbidity centrates is needed prior to depth filtration. A flocculation or precipitation operation is performed following centrifugation to clarify the centrate in a quick, efficient, and cost-effective process. Clarifying the centrate prior to depth filtration, instead of prior to centrifugation as is common for fed-batch culture (a) avoids the flocculation-related cell lyses that can deleteriously affect product quality, (b) potentially avoids or minimizes flocculation-related cell culture or cell sediment rheology behavior changes affecting centrifugation, and c) reduces any depth filtration membrane area required to produce clarified centrate that can be loaded onto a chromatography column. Unlike fed-batch culture harvest methods where flocculation is commonly performed as a first step followed by centrifugation to remove the flocculated material, adding flocculant directly to the perfusion culture broth having a PCV of at least 16% prior to centrifugation does not result in a viable commercial scale harvest operation. The flocculated material impedes continuous solids discharge centrifugation.

Flocculant may be added into centrate in batch mode or in-line continuous mode. A pre-determined amount of flocculant solution is added to the centrate pool to achieve the target flocculant concentration. Alternatively, the flocculant solution may be added at a pre-determined ratio of flocculant flow rate and centrate flow rate to achieve the target flocculant concentration.

Exemplary flocculants suitable for use in the methods herein are cationic flocculants. As many process- and product-related impurities are negatively charged in cell culture media, cationic flocculants are used to reduce/remove them from the centrate. Exemplary cationic flocculants include polymers of diallyl dimethyl ammonium chloride, e.g., poly diallyl dimethyl ammonium chloride (pDADMAC), and its monomer, diallyl dimethyl ammonium chloride (DADMAC), as well as poly ethyleneimine (PEI), polyacrylamide (PAA), and chitosan (U.S. Pat. No. 9,371,554, and McNerney et al., mAbs 7:2 413-427 (2015) incorporated herein by reference). Additional exemplary flocculants include simple acids including organic acids such as caprylic acid or octanoic acid (WO 2010/151632, incorporated herein by reference), divalent cations, polycationic polymers such as polyethyleneimine (PEI), non-ionic polymers such as polyethylene glycol (PEG) (U.S. Pat. No. 9,371,554, and McNerney et al., mAbs 7:2 413-427 (2015) incorporated herein by reference), and polyalkylene glycols (and transition metals such as zinc, US Pat. Pub. No. 2009/0292109 incorporated herein by reference), non-ionic surfactants, and stimulus-responsive polymers (Kang et al., Biotechnol. Bioeng. 110:2928-2937 (2013), incorporated herein by reference), such as benzylated poly(allylamine). A “stimulus-responsive polymer” or “Smart polymer” is a compound comprising a plurality of chemical sub-units (monomers) that is sensitive to at least one trigger from the external environment, including temperature, light, electrical or magnetic fields, and chemicals, that results in a detectable change in the properties of the polymer such as solubility and the capacity to induce aggregation. Such agents are useful in methods according to the disclosure as flocculating compounds or attached to an insoluble matrix such as silica beads or the eXtreme-Density (XD) cell culture process (Schirmer et al., Bioproc. Intl. 8:32-39 (2010), incorporated herein by reference), to aid in centrate clarification.

In some embodiments the flocculant is poly(diallyldimethylammonium chloride) (pDADMAC), or its monomer, diallyl dimethyl ammonium chloride (DADMAC). In some embodiments, pDADMAC is added to the centrate at 0.04-0.15% (w/w). In some embodiments the pDADMAC or DADMAC is added to the centrate at 0.05% (w/w). Flocculation is performed at 8-12° C., with flocculation typically performed at 10° C. In some embodiments flocculation is performed at about 8° C., 8.5° C., 9° C., 9.5° C., 10° C., 10.5° C., 11° C., 11.5° C., or 12° C. In some embodiments flocculation is performed at about 10° C. After flocculant addition, the mixture is allowed to mix for 30 minutes before beginning depth filtration. In some embodiments, the flocculated mixture is stored for up to 4 hours at 8-12° C., e.g., 10° C., with mixing. In some embodiments, the entire harvest process from cooling of bioreactor contents through depth filtration of the centrate is performed at 10° C. or performed with fluid that is at 10° C. In some embodiments the centrifugation, flocculation, and depth filtration are performed with 10° C. fluid.

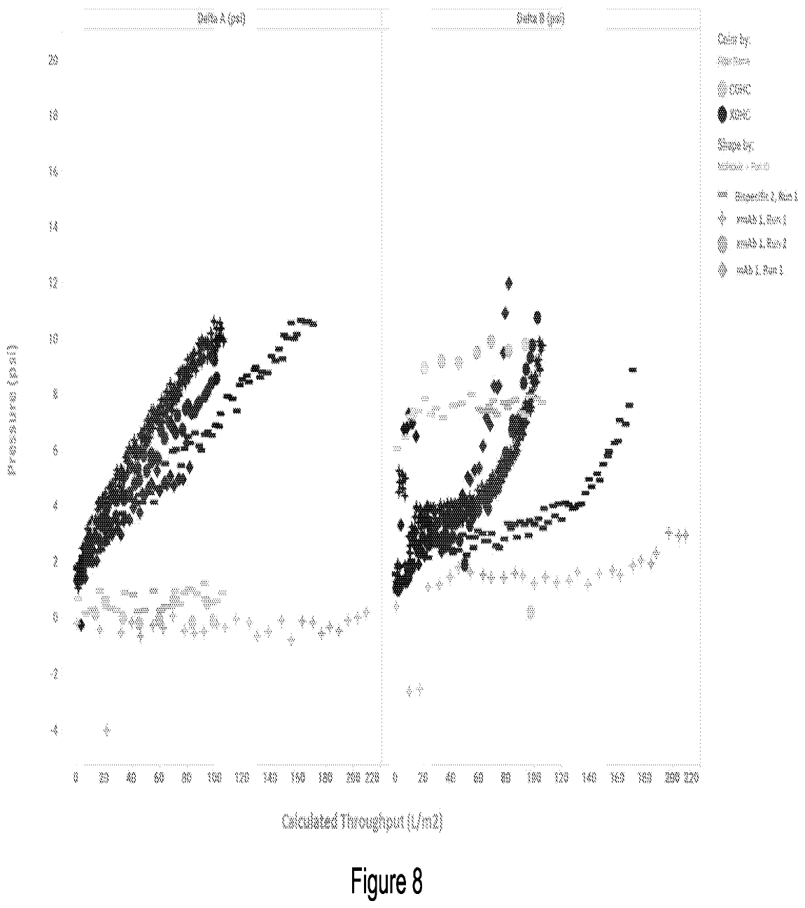

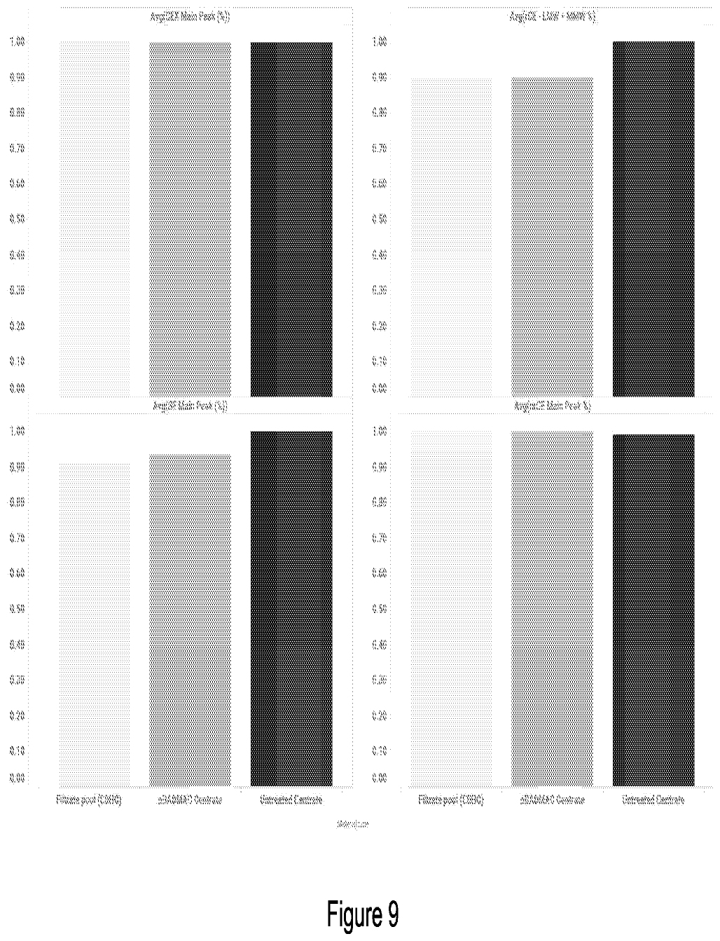

Depth filters make use of a variety of porous filtration media, for example, diatomaceous earth, cellulosic, and/or synthetic fibers, creating a three-dimensional network of pores that collectively establish tortuous paths that materials must take to pass through the filter. Particulate matter from cell culture broths, e.g., cells, cell debris, byproducts, and both product- and process-related impurities can rapidly foul filters by plugging the pores or the surface of the filter. As would be known in the art, the rate and degree of filter fouling or clogging is influenced by the cell debris particle quantity and size distribution as well as the depth filter media chemistry and pore size. Disclosed herein are data establishing that, when untreated centrate is paired with X0HC, or even a larger pore size such as C0HC, there was almost immediate clogging (see FIG. 8). Flocculation ensures the formation of larger molecular aggregates, which reduces the cell debris particle quantity and increases the particles size, reducing the incidence of fouling or clogging especially when paired with a suitable filter type (e.g., C0HC, C0SP, DL75, DL60). Selection of a suitable depth filter type is based on empirical optimization of flocculation, depth filter media chemistry, and depth filter media pore size range. In particular, the pore size is matched to the size of cell debris you have in solution. Without flocculation, particles were found in a size range less than 1-2 μm. With flocculation, particles were found in the 50-100 μm range. Overall, there were fewer particles after flocculation as well. Small and large particles can clog the filter. The worst performance was observed when the selected pore size of the depth filter dose did not match the size of particles in solution, which is why a C0HC filter with untreated centrate has worse performance than a X0HC filter type. As shown in FIG. 1, a wide range of depth filter types shows increased depth filtration loading relative to an X0HC control. The X0HC control was selected for untreated centrate because it had previously been shown to provide the best depth filtration capacity for untreated centrate. Increased depth filtration capacity reduces the quantity of depth filters required filtration of a fixed centrate volume, reducing the filter area footprint in the manufacturing facility.

Suitable depth filters are composed of porous matrices of synthetic or cellulosic fibers, optionally attached to charged resins used to attract and retain oppositely charged filter aids, as would be known in the art. Some exemplary depth filters incorporate diatomaceous earth as an inorganic filter aid. Depth filters may also contain binders. For all depth filters, including cellulosic depth filters, the thicker the material, e.g., the cellulosic fiber mat or material, the more sinuous and complicated is the path through the filter material, which results in particles of various sizes being captured and removed from the fluid being clarified. Cellulosic depth filters typically have a net positive charge, which also provides a basis for adsorptive removal of negatively charged impurities.

The methods of the disclosure include exposing a clarified centrate from a continuous solids discharge disc stack centrate to depth filtration to remove impurities following the flocculation step as disclosed herein. The depth filtration operation may include one or more depth filters of the same or different types. Depth filters suitable for use in the methods of the disclosure include, but are not limited to, cellulose, synthetic fiber meshes or a combination of both, as well as depth filters made with pre-treated filtration matrix as described in Singh et al., Biotechnol. Bioeng. 110:1964-1972 (2013), incorporated herein by reference. Based on the current small-scale depth filtration data as disclosed herein, exemplary depth filtration types appropriate for use with flocculated centrate in the harvest methods of the disclosure include C0HC (0.2-2 um nominal pore size distribution), C0SP (0.2-2 um nominal pore size distribution), DL75 (1-14 um nominal pore size distribution) and DL60 (0.6-10 um pore size distribution). Those of skill in the art can identify other filter media suitable for use in the disclosed methods using no more than routine technical skill. Expanding on the preceding observations, exemplary depth filters include a cellulosic depth filter, a MILLISTAK+® D0HC filter, a MILLISTAK+® C0HC filter, a MILLISTAK+® C0SP filter, a SARTOCLEAR® DL60 filter, a SARTOCLEAR® DL75 filter, a Clarisolve 20MS filter, a Clarisolve 40MS filter, a Clarisolve 60HX filter, a 3M™ Zeta Plus™ Filter, diatomaceous earth, and a 3M™ Emphaze™ AEX Hybrid Purifier. All of these filter media will provide useful purification without unacceptable losses in yield, but as noted herein, filter media loading will be reduced if filter media pore size is not selected appropriately based on particle sizes in the centrate being filtered. Based on the data disclosed herein, it is expected that multi-layer filters will be advantageous in purifying target proteins obtained in high yield, but multi-layer filters are not a requirement for the methods according to the disclosure. The experiments relating to the depth filtration component of the purification methods are designed to yield data useful in achieving high loading levels in order to minimize the number of filters needed to clarify the centrate sufficiently to pass through any subsequent downstream filters, such as a Protein-A guard filter (typically 0.2 μm pore size), while preserving a high step yield.

In addition, the depth filtration may be followed by one more sterile or bioburden reduction filters (e.g., Millipore SHC filters, Sartopore 2 filters (Sartorius), and Pall filters) having pore sizes no larger than 0.2 μm to ensure bioburden reduction. Upon completion of depth filtration, the purified centrate is held at 2-8° C., typically for no more than 72 hours. The hold may be reduced depending on molecule stability.

The disclosure further contemplates methods combining the harvest process with post-harvest recovery steps known in the art, such as target biomolecule recovery, e.g., affinity-based recovery which, for immunoglobulins and immunoglobulin-like targets, may be based on binding of the target affinity chromatography materials that make use of Staphylococcus proteins, such as Protein A, as well as chromatographic fractionations and polishing steps wherein the chromatographic medium can be in any form, including beads in column form, and the fractionations and polishing steps can rely on any discriminatory property of the target, such as size (e.g., size-exclusion chromatography), affinity, charge (e.g., anionic or cationic exchange chromatography, hydrophobicity (e.g., hydrophobic interaction chromatography), multimodal or mixed-modal (combination of different modes) or any property known to be useful in discriminating among molecules using a chromatographic medium. In simple terms, the disclosure provides any methods for purifying a target biomolecule, e.g., a biologic such as an antibody, that has been harvested using methods disclosed herein.

The disclosure provides for separation of recombinant proteins from cell culture broth in a batch harvest stream from a cell culture bioreactor. The culture is harvested from the bioreactor when a predetermined parameter is met, e.g., culture duration, titer, viable cell density, or packed cell volume. The bioreactor is cooled to a temperature of 12° C. or less. The cell culture broth is then harvested directly from the bioreactor or collected into a holding tank and held at 10-12° C. The harvest cell viability, viable cell density (VCD), packed cell volume and/or baseline turbidity of the cultures are determined using conventional techniques known in the art.

The disclosure provides for separation of recombinant proteins from cell culture broth in a periodic harvest stream from a perfusion culture process, where the cell culture broth is passed through a surge tank/vessel prior to, or following, the continuous solids discharge disc stack centrifuge separating the solid materials (heavy phase) from the liquid supernatant (light phase or centrate). The centrate is then exposed to a flocculation/precipitation step prior to being loaded onto one or more depth filters, and optionally also to one or more sterile filters. The eluate is then collected into a holding tank and stored at 4° C. or directly subjected to a purification chromatography operation.

The disclosure provides for continuous separation of recombinant proteins from continuous cell cultures, where batches of cell culture broth are passed through a continuous solids discharge disc stack centrifuge separating the solid materials from the cell culture supernatant (centrate). In some embodiments, the solids may be recirculated or re-introduced into the bioreactor. The centrate is collected in a harvest pool and subjected to the remaining steps of the harvesting methods described herein. In some embodiments, the methods involve continuous harvest directly from a working bioreactor, and some embodiments provide for an in-line cool down.

Harvest operations described herein can be combined with additional harvest strategies, including additional centrifugation, such as disc stack centrifugation; filtration, including tangential flow filtration, microfiltration, ultrafiltration, and depth filtration; sedimentation methods, and chromatography media-based separations, typically using columns that include anion- and/or cation-exchange, affinity-based exchange (e.g., immunoaffinity exchange), molecular sieving, and/or polishing chromatography. Any purification or clarification protocol known in the art to be applicable to cell culture materials, including cell culture supernatant and fluids containing cellular materials, is comprehended by the methods disclosed herein. In a typical operation, separation via continuous solids discharge disc stack centrifugation is the first step following removal of cell culture from a bioreactor (or a surge tank or holding tank useful in batch-mode separations). Following the centrifugation, flocculation, and filtration harvest steps described herein, the purification or clarification protocol may include any combination of downstream filters and/or columns and/or other forms of centrifugation useful in separating biomolecules such as proteins.

Beyond the foregoing technologies, the methods of the disclosure contemplate a wide variety of chromatographic steps involved in separating, or clarifying, biomolecules (e.g., proteins) of interest, including size exclusion chromatography, ion exchange chromatography, hydrophobic interaction chromatography, multimodal or mixed-modal chromatography, and affinity chromatography, such as immunoaffinity chromatography. Various media may be used in a variety of configurations designed to contribute to biomolecule, e.g., protein, separations in batch and continuous modes of purification.

The method of the present disclosure contemplates a production process whereby cells are cultured in three or more distinct phases. Each phase can be conducted in its own bioreactor vessel or other vessel suitable for cell culture. Alternatively, more than one phase can be conducted in a common vessel. For a commercial process for producing proteins using mammalian cells, there are commonly multiple, for example, at least about 2, 3, 4, 5, 6, 7, 8, 9, or 10 growth phases/stages that occur in different culture vessels preceding the final N production phase. For example, cells may be cultured in one or more growth phases prior to the N-1 seed stage, and cultured in one or more N-1 bioreactors. The duration of the N-1 stage can range from, e.g., 7 to 14 days, and can be designed to maintain the cells in exponential growth prior to inoculation of a production (N) bioreactor. In some embodiments, the cells from the N-1 bioreactor(s) are transferred to a (N) production bioreactor and grown under conditions that maximize protein production. In other embodiments, there is no transfer, i.e., the N-1 and N production stages occur in the same bioreactor.

“Cell” or “Cells” include any prokaryotic or eukaryotic cell. Cells can be either ex vivo, in vitro or in vivo, either separate or as part of a higher structure such as a tissue or organ. Cells include “host cells”, which may be genetically engineered to express a protein of commercial or scientific interest. Host cells are typically derived from a lineage arising from a primary culture that can be maintained in culture for an unlimited time. A host cell can be a prokaryotic cell (for example, E. coli) or eukaryotic cell (for example, yeast, insect, or animal cells (e.g., CHO)). Host cells cultured under appropriate conditions express the protein of interest which may be subsequently collected from the culture medium (if the host cell secretes it into the medium), collected directly from the host cell producing it (if the protein is not secreted), or collected from both sources. The selection of an appropriate host cell will depend upon various factors, such as desired expression levels, protein modifications that are desirable or necessary for activity (such as glycosylation or phosphorylation) and ease of folding into a biologically active molecule.

Genetically engineering the host cell involves transfecting, transforming or transducing the cells with a recombinant polynucleotide molecule, and/or otherwise altering (e.g., by homologous recombination and gene activation or fusion of a recombinant cell with a non-recombinant cell) to cause the host cell to express a desired recombinant protein. Methods and vectors for genetically engineering cells and/or cell lines to express biomolecules (e.g., proteins) of interest are well known to those of skill in the art. Expression systems and constructs in the form of expression vectors such as plasmids or transcription or expression cassettes that comprise one or more polynucleotides encoding a biomolecule (e.g., protein) of interest, such as those identified herein, are provided, as well as host cells comprising such expression systems or constructs. As used herein, “vector” means any molecule or entity (e.g., nucleic acid, plasmid, bacteriophage, transposon, cosmid, chromosome, virus, virus capsid, virion, naked DNA, complexed DNA and the like) suitable for use to transfer and/or transport protein-encoding information into a host cell and/or to a specific location and/or compartment within a host cell. Vectors can include viral and non-viral vectors, and non-episomal mammalian vectors. Vectors may be expression vectors, for example recombinant expression vectors, or cloning vectors. The vector may be introduced into a host cell to allow replication of the vector itself and thereby amplify the copies of the polynucleotide contained therein. The cloning vectors may contain sequence components that generally include, without limitation, an origin of replication, promoter sequences, transcription initiation sequences, enhancer sequences, transposons/transposases, and selectable markers. These elements may be selected as appropriate by a person of ordinary skill in the art, and one or more of each selected component may be included within a cloning vector.

Preferably, host cells are eukaryotic cell, such as a mammalian cell. Any mammalian cell suitable for recombinant protein expression is appropriate for use in the context of the disclosure. Suitable mammalian cells include, but are not limited to, Chinese hamster ovary (CHO) cells, human embryonic kidney (HEK) cells, murine myeloma (NS0, Sp2/0) cells, baby hamster kidney (BHK) cells, human embryonic kidney (293) cells, fibrosarcoma (HT-1080) cells, human embryonic retinal (PER.C6) cells, hybrid kidney and B cells (HKB-11), CEVEC's amniocyte production (CAP) cells, human liver (HuH-7) cell, and any other cells that are used or suitable for use in clinical and/or commercial manufacturing. The most commonly used cell lines are from CHO cells. CHO cells are widely used to produce complex recombinant proteins. The dihydrofolate reductase (DHFR)-deficient mutant cell lines (Urlaub et al. (1980), Proc Natl Acad Sci USA 77: 4216-4220), DXB11 and DG-44, are desirable CHO host cell lines because the efficient DHFR selectable and amplifiable gene expression system allows high level recombinant protein expression in these cells (Kaufman R. J. (1990), Meth Enzymol 185:537-566). The glutamine synthetase (GS)-knockout CHOK1SV cell lines, making use of glutamine synthetase (GS)-based methionine sulfoximine (MSX) selection are also widely used. Also included are CHOK1 cells (ATCC CCL61).

Critical attributes and performance parameters can be measured to better inform decisions regarding performance of each step during manufacture. These critical attributes and parameters can be monitored real-time, near real-time, and/or after the fact. Key critical parameters such as media components that are consumed (such as glucose), levels of metabolic by-products (such as lactate and ammonia) that may accumulate in the culture, as well as those related to cell maintenance and survival, such as dissolved oxygen content can be measured during the cell culture. Critical attributes such as specific productivity, viable cell density, pH, osmolality, appearance, color, aggregation, cell count, packed cell volume, percent yield and titer may be monitored during appropriate stages in the manufacturing process. Process and product impurities may also be monitored throughout the manufacturing process.

By “culture” or “culturing” is meant the growth and propagation of cells outside of a multicellular organism or tissue. Suitable culture conditions for mammalian cells are known in the art. Cell culture media and tissue culture media are interchangeably used to refer to media suitable for growth of a host cell during in vitro cell culture. Typically, cell culture media contains a buffer, salts, energy source, amino acids, vitamins and trace essential elements. Any media capable of supporting growth of the appropriate host cell in culture can be used. Cell culture media, which may be further supplemented with other components to maximize cell growth, cell viability, and/or recombinant protein production in a particular cultured host cell (including components effective in controlling the timing of recombinant protein production), are commercially available and include RPMI-1640 Medium, RPMI-1641 Medium, Dulbecco's Modified Eagle's Medium (DMEM), Minimum Essential Medium Eagle, F-12K Medium, Ham's F12 Medium, Iscove's Modified Dulbecco's Medium, McCoy's 5A Medium, Leibovitz's L-15 Medium, and serum-free media such as EX-CELL™ 300 Series, among others, which can be obtained from the American Type Culture Collection or SAFC Biosciences, as well as other vendors. Cell cultures can also be supplemented with independent concentrated feeds of particular nutrients which may be difficult to formulate or are quickly depleted in cell cultures. Such nutrients may be amino acids such as tyrosine, cysteine and/or cystine (see e.g., WIPO Publication No. 2012/145682). Cell culture media can be serum-free, protein-free, growth factor-free, and/or peptone-free media. Cell culture may also be enriched by the addition of nutrients at greater than their usual, recommended concentrations. In preferred embodiments, cell culture media is tailored to the growth of host cells to cell densities greater than 10⁶ cells/ml, i.e., to cell densities approaching, reaching, or exceeding 10⁸ cells/ml.

Perfusion feed media may be formulated or supplemented to achieve a concentration of at least 5 g/L of a non-ionic block copolymer when passing the cell culture through a hollow fiber filter having a pore size or molecular weight cut off (MWCO) that does not retain the recombinant protein in the bioreactor (WO 2015/188009).

Various media formulations can be used during the life of the culture, for example, to initiate the culture, to facilitate the transition from one stage (e.g., the growth stage or phase) to another (e.g., the production stage or phase) and/or to optimize conditions during cell culture (e.g., concentrated media provided during perfusion culture). A basal medium formulation containing essential media components, is typically used to initiate the cell culture. A growth medium formulation can be used to promote cell growth and minimize protein expression. A production medium formulation can be used to promote production of the biomolecule (e.g., protein) of interest and maintenance of the cells, with minimal new cell growth). A feed medium, typically a medium containing more concentrated components such as nutrients and amino acids, which are consumed during the course of the production phase of the cell culture, may be used to supplement and maintain an active culture, particularly a culture operated in perfusion mode. Such a concentrated feed medium can contain most of the components of the cell culture medium at, for example, about 5×, 6×, 7×, 8×, 9×, 10×, 12×, 14×, 16×, 20×, 30×, 50×, 100×, 200×, 400×, 600×, 800×, or even about 1000× of their normal amount or concentration.

Cell growth can be limited or arrested during a cell culture run, particularly during a production phase. Such methods include, temperature shifts, pH shifts, use of chemical inducers of protein production and cell cycle inhibitors, nutrient limitation or starvation, either alone or in combination. For example, a temperature shift may be used to transition from a growth phase to a production phase. The growth phase may occur at a first temperature from about 35° C. to about 38° C., and the production phase may occur at a second temperature from about 29° C. to about 35° C., optionally from about 30° C. to about 35° C. or from about 30° C. to about 34° C. Optionally about 32° C. to about 33° C. In addition, a growth phase may occur at a higher pH than a production. A pH shift may be used separately or in combination with a temperature shift and/or addition of chemical inducers.

Another method to maintain cells at a desired physiological state is to induce cell growth-arrest by exposure of the cell culture to low L-asparagine conditions and/or asparagine starvation (see e.g., WIPO Publication No. WO 2013/006479). Cell growth-arrest may be achieved and maintained through a culture medium that contains a limiting concentration of L-asparagine and maintaining a low concentration of L-asparagine in the cell culture. Maintaining the concentration of L-asparagine at 5 mM or less can be used to induce and maintain cells in a growth-arrested state whereby productivity is increased.

In addition, chemical inducers of protein production, such as caffeine, butyrate, and hexamethylene bisacetamide (HMBA), may be added before, at the same time as, and/or after a temperature shift. If inducers are added after a temperature shift, they can be added from one hour to five days after the temperature shift, optionally from one to two days after the temperature shift. Cell cycle inhibitors, compounds known or suspected to regulate cell cycle progression and the associated processes of transcription, DNA repair, differentiation, senescence and apoptosis related to this are also useful to induce cell growth-arrest. Cell cycle inhibitors that interact with the cycle machinery, such as cyclin-dependent kinases (CDKs) are useful as are those molecules that interact with proteins from other pathways, such as AKT, mTOR, and other pathways that affect, directly or indirectly, the cell cycle.

The method of the present disclosure can be used as part of a larger production process whereby cells are cultured in three or more distinct phases. Each phase can be conducted in its own bioreactor vessel or other vessel suitable for cell culture. Alternatively, more than one phase can be conducted in a common vessel. Suitable vessels include, fluidized bed bioreactors, hollow fiber bioreactors, roller bottles, shake flasks, or stirred tank bioreactors, with or without microcarriers. In general terms as used herein, a bioreactor is any container used for cell culture.

For use in the methods described herein cell cultures are operated in perfusion mode. Mammalian cells, such as CHO cells, may be cultured in bioreactors at a smaller scale of less than 100 ml to less than 1000 ml. Alternatively, larger scale bioreactors at 1000 ml to over 2,000 liters can be used. In one embodiment 1 liter to 2000 liters are used. In one embodiment the bioreactor is 10 liters to 2000 liters. In one embodiment the bioreactor is 10 liters to 100 liters. In one embodiment the bioreactor is 30 liters to 50 liters. In one embodiment the bioreactor is 30 liters to 2000 liters. In one embodiment the bioreactor is 100 liters to 2000 liters. In one embodiment the bioreactor is 500 liters to 2000 liters. In one embodiment the bioreactor is 1000 liters to 2000 liters. Larger scale cell cultures, such as for clinical and/or commercial scale biomanufacturing of protein therapeutics, may be maintained for weeks or months, while the cells produce the desired protein(s).

In some embodiments, the method of the disclosure is conducted using single-use bioreactors, also known as disposable bioreactors, which utilize disposable bags instead of traditional stainless steel culture vessels. Use of single-use technology minimizes infrastructure requirements associated with traditional cell culture, such as steel/glass commercial-scale vessels and associated machinery. Single-use bioreactors provide flexibility to the manufacturing process, and site assembly, reconfiguration, sterilization, and validation are faster, easier, and less costly than traditional built-in-place stainless steel cell culture plants. Single-use bioreactors comprise disposable, plastic sterile bags supported by a non-disposable support structure. The culture is agitated by a stirrer within the bag or by rocking, air and oxygen spargers are also supplied as well as sensors to measure and adjust various parameters of the culture, such as pH, temperature, oxygen, cell density, and the like. Single-use bioreactors are commercially available, for example, Bio STR®, Satorius, Göttingen Germany; MOBIUS®, Millipore, Burlington, MA; XCELLEREX®, Cytiva, Marlborough, MA. In some embodiments, it is expected that cultures of 3 kL or more will be grown in bioreactors of suitable size.

The bioreactor system maintains conditions within the bioreactor to support cell culture. Suitable culture conditions for mammalian cells are known in the art. See, e.g., Animal cell culture: A Practical Approach, D. Rickwood, ed., Oxford University Press, New York (1992). By “running” a bioreactor system is meant maintaining conditions in the bioreactor system to support cell culture. A bioreactor “run” typically comprises the steps of inoculating a prepared bioreactor with a seed culture, subjecting the cells to one or more growth phase and/or production phases until a predetermined parameter (time, viable cell density, packed cell volume) is met and then harvesting the contents of the bioreactor.

“Culture” or “Culturing” refers to maintaining a cell in culture medium under conditions suitable for the survival and/or proliferation of the cell apart from a multicellular organism or tissue, and for producing the protein product. Cell cultures are typically operated in batch, fed batch, or perfusion modes. In batch mode, a fixed amount of culture medium and cells are added to the bioreactor at the start of the run. Over the course of the culture, the media volume in the reactor remains constant while the nutrient content of the media decreases and waste and byproduct content increases. The cell concentration increases over the course of the run and will plateau and decline as nutrient content is depleted and waste products increase. Fed batch culture begins with an inoculation of cells and a fixed amount of culture media. Unlike batch culture, the volume of media in the bioreactor increases as concentrated nutrients are added over the course of the culture. Like batch culture, the waste and byproduct content increase over the course of the run and will plateau and decline as nutrient content is depleted and waste products increase.

Perfusion culture, like batch and fed-batch culture, begins with a fixed inoculation of cells and culture media. Unlike fed-batch culture, the culture is initiated at or near the final working volume of the reactor. Also unlike fed-batch culture, while fresh feed medium is added to the bioreactor, an equivalent amount of spent media is perfused or withdrawn from the bioreactor via a retention device. In the case of the methods described herein, the perfusion is a continuous perfusion. A retention device, such a tangential flow filtration (TFF) system or an alternating tangential flow (ATF) system may be used to remove the spent media and unwanted products from the bioreactor. Alternatively, acoustic wave technology or cell settling technology may be used to remove spent media and unwanted products.

ATF relies on use of a recirculation means, most typically a diaphragm pump, to move cell culture back and forth through a module (e.g., through hollow-fiber filter modules) to allow removal of spent media while retaining cells in the bioreactor. See, e.g., U.S. Pat. No. 6,544,424; Furey (2002) Gen. Eng. News. 22 (7), 62-63. The benefit of ATF is a cleaning effect on the filter that is induced by the alternating flow. An exemplary method of the disclosure comprises running an (N) production bioreactor using an ATF perfusion system.

Typically, hollow fiber filters are used in the ATF system (although this is not required). When the cell culture, including cell culture media, cells (whole and lysed), soluble expressed recombinant proteins, host cell proteins, waste products and the like, are introduced to the filter, depending on the pore size or molecular weight cutoff (MWCO), the hollow fiber material may retain certain cell culture components (in addition to the cells, themselves) on the lumen side (inside) and allow certain components to pass through the filter (permeate) based on the pore size or molecular weight cutoff of the hollow fiber material. The material that is retained (retentate) is returned to the bioreactor. Fresh perfusion culture media is added to the bioreactor and permeate is withdrawn from the filter at predetermined intervals or continuously to maintain a desired or constant bioreactor volume. The permeate can be discarded, stored in holding tanks, bags or totes or transferred directly to another unit operation, such as a harvest operation.

Exemplary filters (e.g., ultrafilters and microfilters) include, but are not limited to, Millipore prostracks, Cytiva hollow-fiber filters in a number of pore size ranges, Repligen filters of various pore sizes, Spectrum hollow-fiber filters of various pore sizes, and Ashai Kasei hollow-fiber filters.

Typically, hollow fiber filters are used in perfusion culture retention systems. The hollow fibers, in various aspects, have inner diameters of about 0.5 mm to about 1 mm, and may be of any suitable length (e.g., about 30 cm to about 110 cm). Hollow fibers for microfiltration typically have a pore size ranging from 0.1 μm to 10 μm or a molecular weight cut off of 500 to 750 kDa or more and can be used to allow the protein (e.g., a monoclonal antibody or an engineered antibody-like protein) to pass through into the permeate. Ultrafiltration hollow fibers typically have a pore size range of 0.01 μm to 0.1 μm or a molecular weight cut off of 300 kDa or less and can be used to retain the desired protein in the retentate for return to the bioreactor. This can be used, for example, to concentrate a recombinant protein product for harvest. Such filters are available commercially, such as Xampler, (Cytiva, Marborough, MA), Midikros (Spectrum Laboratories, Inc, Dominguez, CA.), XCell ATF®, Repligen, Waltham, MA). The temperature of the culture broth exiting the bioreactor is controlled, with the temperature typically maintained at 10-12° C. The culture broth is removed from the bioreactor to the continuous solids discharge disc stack centrifuge or a surge tank using a pump. As would be understood, the centrifuge itself also has a pump.

Cell culture fluid may be drawn out of the bioreactor and into the filter module by a pumping system, which passes the cell culture through or along a filter (e.g., through the lumen side of the hollow fiber). Examples of cell pumping systems include peristaltic pumps, double diaphragm pumps, low shear pumps (Levitronix™ pumps, Zurich, Switzerland), and alternating tangential flow systems (ATF™, Repligen, Waltham, MA). The permeate may be drawn from the filters by use of peristaltic pumps. In the examples provided herein, perfusion is accomplished by use of an alternating tangential flow system.

Medium exchange in the bioreactor is expressed as vessel volumes per day (VVD) or bioreactor volumes per day (BV/d). The exchange rate may be minimized, such as by being set at about 0.25 BV/d, but is more frequently set at a value of at least 1 BV/d; typically, the rate is set at a value of at least 2 BV/d to 3 BV/d.

Following expression in the perfusion culture, the target protein of interest is then harvested according to the methods of the disclosure. The harvesting process clarifies or purifies, or partially clarifies or purifies, the target protein away from at least one impurity with which it is found in culture broth, such as remaining cell culture media, cell extracts, cells, cell debris, undesired cell or medium components, product- and/or process-related impurities. The harvested recombinant protein of interest can be stored in surge tanks, holding tanks, bags, or other containers that may be adapted to provide feed to a chromatography column skid. The harvested recombinant protein of interest may also be provided directly to a chromatography column skid as an eluate stream. At harvest, the PCV of the perfusion culture is at least 16%. In one embodiment, the PCV of the perfusion culture is at least 18%. In one embodiment, the PCV of the perfusion culture is about 20-about 30%. In one embodiment, the perfusion culture is about 16%. In one embodiment, the PCV of the perfusion culture is about 18%. In one embodiment, the PCV of the perfusion culture is about 19%. In one embodiment, the PCV of the perfusion culture is about 20%. In one embodiment, the PCV of the perfusion culture is about 21%. In one embodiment, the PCV of the perfusion culture is about 22%. In one embodiment, the PCV of the perfusion culture is about 23%. In one embodiment, the PCV of the perfusion culture is about 24%. In one embodiment, the PCV of the perfusion culture is about 25%. In one embodiment, the PCV of the perfusion culture is about 26%. In one embodiment, the PCV of the perfusion culture is about 27%. In one embodiment, the PCV of the perfusion culture is about 28%. In one embodiment, the PCV of the perfusion culture is about 29%. In one embodiment, the PCV of the perfusion culture is about 30%. In one embodiment, the PCV of the perfusion culture is about 30-about 35%. In one embodiment, the PCV of the perfusion culture is about 16%-45% Recovery and further purification of the target protein of interest may be accomplished by downstream unit operations.

Downstream operations purify and polish the target protein. The downstream operations make use of capture chromatography that includes resins and/or membranes containing agents that will bind and/or interact with at least one desired protein, impurity, or contaminant. Examples of capture chromatography include affinity chromatography, size exclusion chromatography, ion exchange chromatography (IEX) such as cation exchange (CEX) and anion exchange (AEX) chromatography, hydrophobic interaction chromatography (HIC), multimodal or mixed-modal (MMC), immobilized metal affinity chromatography (IMAC), and the like. Such materials are known in the art and are commercially available.

Affinity chromatography is commonly used in biomanufacturing processes as an initial capture step to isolate and concentrate target proteins, such as recombinant proteins, of interest from crude or clarified material. Examples of such affinity chromatography materials include those that make use of Staphylococcus proteins such as Protein A, Protein G, Protein A/G, and Protein L; substrate-binding capture mechanisms; antibody- or antibody fragment-binding capture mechanisms; aptamer-binding capture mechanisms; cofactor-binding capture mechanisms; and the like. Immobilized metal affinity chromatography can be used to capture proteins that have or have been engineered to have affinity for metal ions. Protein A is highly selective for a wide range of antibody and antibody-like proteins and robust removal of process-related impurities and high yields are relied upon as a first line, bulk purification process. Protein A material is available commercially from a number of vendors. For example, MABSELECT™ SURE Protein A, Protein A Sepharose FAST FLOW™ (Cytiva, Marborough, MA), PROSEP-A™ (Merck Millipore, U.K), and TOYOPEARL™ 650M Protein A (TosoHass Co., Philadelphia, PA).

Intermediate and/or polishing unit operations make use of various chromatography methods for the continued purification of the protein of interest and clearance of contaminants and impurities such as DNA, host cell proteins, product-related impurities, variant products and aggregates, viruses (such as by virus adsorption), and the like. These chromatography unit operations make use of resins and/or membranes containing agents that can be operated in a variety of configurations, such as flow-through mode where the protein of interest is contained in the eluent and the contaminants and impurities are bound to the chromatography medium; frontal or overloaded chromatography mode where a solution containing the protein of interest is loaded onto a column until adsorption sites are occupied and the species with the least affinity for the stationary phase (the protein of interest) starts to elute; and bind-and-elute mode, where the protein of interest is bound to the chromatography medium and eluted after the contaminants and impurities have flowed through or been washed off the chromatography medium, or any other method for achieving some degree of purification of a target protein via chromatography. As indicated above, examples of such chromatography methods include ion exchange chromatography (IEX), such as anion exchange chromatography (AEX) and cation exchange chromatography (CEX); hydrophobic interaction chromatography (HIC); mixed modal or multimodal chromatography (MM), hydroxyapatite chromatography (HA); reverse phase chromatography and gel filtration, among others.

Multiple chromatography unit operations, usually one, two, or three, each typically performing a different function or operating in different modes, are combined depending on the requirements of the manufacturing process. Ion exchange chromatography, based on electrostatic interactions between charged surfaces, separates proteins of interest from impurities based on differential rates of adsorption and desorption reflective of the level of electrostatic attraction of the protein (or impurity) to the chromatography medium. Cation exchange chromatography refers to chromatography performed on a solid-phase medium that is negatively charged and has cations available for exchange with cations in an aqueous solution passed over or through the solid phase. The charge may be provided by attaching one or more charged ligands to the solid phase, e.g., by covalent linking. Alternatively, or in addition, the charge may be an inherent property of the solid phase (e.g., as is the case for silica, which has an overall negative charge). Cation exchange chromatography is typically run in bind-and-elute mode, and the high pI of many proteins of interest enable binding to the chromatography material. Cation exchange chromatography may also be run in flow-through mode. CEX chromatography is typically used to remove high molecular weight (HMW) contaminants, process-related impurity, and/or viral clearance. Commercially available cation exchange media are available and include, but are not limited to, sulphopropyl (SP) functional groups immobilized on agarose (e.g., SP-SEPHAROSE FAST FLOW™, SP-SEPHAROSE FAST FLOW XL™ or SP-SEPHAROSE HIGH PERFORMANCE™, CAPTO S™, CAPTO SP ImpRes™, CAPTO S ImpAct™ (Cytiva), FRACTOGEL-SO3™, FRACTOGEL-SE HICAP™, and FRACTOPREP™ (EMD Merck, Darmstadt, Germany), TOYOPEARL® XS, TOYOPEARL® HS (Tosh Bioscience, King of Prussia, PA), UNOsphere™ (BioRad, Hercules, CA), and S Ceramic Hyper D™ F (Pall, Port Washington, NY), POROS™ (ThermoFisher, Waltham, MA).

Anion exchange chromatography refers to chromatography performed on a solid-phase medium that is positively charged and has anions available for exchange with anions in an aqueous solution passed over or through the solid phase. Anion exchange chromatography is typically run in flow-through mode. Due to the high pI of many therapeutic proteins they do not readily bind to AEX chromatography material. AEX chromatography is used, for example, for viral clearance and impurity removal. Commercially available anion exchange media are available and include, but are not limited to, quaternary amine (Q) immobilized on agarose (e.g. Source 15 Q, Capto™ Q, Q-SEPHAROSE FAST FLOW™ (Cytiva), FRACTOGEL EDM TMAE™, FRACTOGEL EDM DEAE™ (EMD Merck), TOYOPEARL Super Q® (Tosh Bioscience), POROS HQ™, POROS XQ™, (ThermoFisher).

Mixed-mode or multi-mode chromatography (MMC) refers to chromatography performed on a solid-phase medium that makes use of a combination of interaction mechanisms, such as ion exchange (CEX or AEX) and hydrophobic interaction, and others. Commercially available multi-modal chromatography media are available and include Capto™ Adhere (Cytiva).

Hydrophobic interaction chromatography refers to chromatography performed on a solid-phase medium that makes use of the interaction between hydrophobic ligands and hydrophobic residues on the surface of a protein of interest. Commercially available hydrophobic interaction chromatography media includes, but is not limited to, Phenyl Sepharose™ (Cytiva), Tosoh hexyl (Tosoh Bioscience), and Capto™ phenyl (Cytiva).

Hydroxyapatite chromatography refers to chromatography performed on a solid-phase medium that makes use of positively charged calcium and negatively charged phosphate. Depending on the pI of the protein and the pH of the buffer, hydroxyapatite medium can act as a cation or an anion exchanger.

Unit operations engaged in inactivating, reducing and/or eliminating viral contaminants may include processes that manipulate the environment and/or rely on filtration. Viral mitigation measures are critical to ensure the safety of protein therapeutics and may be performed one or more times throughout the downstream purification phases of a harvest operation. Viral contaminants can arise from a variety of sources, including use of reagents of animal origin, adventitious viral contaminants in host cell lines, or system failures at GMP manufacturing sites. Viruses are classified as enveloped and non-enveloped viruses. With enveloped viruses, the envelope allows the virus to identify, bind, enter, and infect target host cells. As such, enveloped viruses are susceptible to inactivation methods. Various methods can be employed for virus inactivation and include heat inactivation/pasteurization, UV and gamma ray irradiation, use of high intensity broad spectrum white light, addition of chemical inactivating agents, surfactants, and solvent/detergent treatments. Surfactants, such as detergents, solubilize membranes and therefore can be very effective in specifically inactivating enveloped viruses. One method for achieving virus inactivation is incubation at low pH (e.g., pH less than 4). Low pH virus inactivation can be followed with a neutralization unit operation that readjusts the virus-inactivated solution to a pH more compatible with the requirements of the following unit operations. Low pH viral inactivation is typically performed following purification of the harvest fluid with affinity chromatography, in particular with affinity chromatography that makes use of a substrate-binding ligand from Staphylococcus aureus, such as Protein A chromatography, since elution from such a chromatography material is usually performed at low pH. Exemplary low pH viral inactivation methods are described in U.S. Patent Application No. 63/168,608 and U.S. Patent Application No. 63/159,217. An exemplary detergent inactivation is described in World International Patent Organization Publication No: WO 2020/190985. Low pH viral inactivation may also be followed by filtration, such as depth filtration, to remove any resulting turbidity or precipitation.

Non-enveloped viruses are more difficult to inactivate without risk to the protein being manufactured and are removed by filtration methods. An exemplary process is described in World International Patent Organization Publication No: WO2020/159838. Viral filtration can be performed using micro- or nano-filters, such as those available from PLAVONA® (Asahi Kasei, Chicago, IL), VIROSART® (Sartorius, Goettingen, Germany), VIRESOLVE® Pro (MilliporeSigma, Burlington, MA), Pegasus™ Prime (Pall Biotech, Port Washington, NY), and CUNO Zeta Plus VR, (3M, St. Paul, Mn). Viral filtration may occur at one or more steps in the downstream operations of a biomanufacturing process. Typically, viral filtration precedes any ultrafiltration or diafiltration (i.e., UF/DF) operation, but may also take place following UF/DF.

Unit operations may also comprise product concentration and buffer exchange of the protein of interest into a desired formulation buffer for bulk storage of the drug substance. Buffer exchange and product concentration can be accomplished using known methods for ultrafiltration and diafiltration. Unit operations to achieve drug product fill/finish can follow. The methods according to the disclosure further optionally comprise concentrating the protein product using ultrafiltration and diafiltration (UF/DF). The purified protein is subjected to an ultrafiltration and diafiltration operation comprising concentrating or diluting the purified protein by ultrafiltration; buffer exchanging the purified concentrated/diluted protein into a desired formulation by diafiltration; and further diluting or concentrating the formulated purified protein by a second round of ultrafiltration until a target protein concentration is achieved. One or more stability-enhancing excipients may optionally be added directly to the UF/DF retentate feed tank containing the formulated purified protein, resulting in a formulated drug substance, or added to the UF/DF eluate pool. Filters for use in a UF/DF operation are well-known and common in the art and are commercially available from a variety of sources. There are many types of materials available, including regenerated cellulose Pellicon (MilliporeSigma, Danvers, MA), stabilized cellulose, Sartocon® Slice, Sartocon® ECO Hydrosart® (Sartorius, Goettingen, Germany), polyethersulfone (PES) membrane, and Omega (Pall Corporation, Port Washington, NY). Multiple filters can be used to the capacity that holders, skids, or the physical set up of the UF/DF system will allow or are needed to achieve the desired objectives of a production process.

Critical attributes and performance parameters of the purified proteins of interest can be measured to better inform decisions regarding performance of each step during manufacture, as is known in the art. These critical attributes and parameters can be monitored real-time, near real-time, and/or after the fact. Key critical parameters such as media components that are consumed (such as glucose), levels of metabolic by-products (such as lactate and ammonia) that accumulate, as well as those related to cell maintenance and survival, such as dissolved oxygen content, can be measured during cell culture. Critical attributes such as specific productivity, viable cell density, pH, osmolality, appearance, color, aggregation, percent yield and titer may be monitored during appropriate stages in the manufacturing process. Monitoring and measurements can be done using known techniques and commercially available equipment.

The pharmaceutical compositions (e.g., solutions, suspensions, and the like), may include one or more of the following: buffers such as neutral-buffered saline, phosphate-buffered saline and the like; carbohydrates such as glucose, mannose, sucrose, dextrans, or mannitol; proteins; polypeptides or amino acids such as glycine; antioxidants; chelating agents such as EDTA or glutathione; adjuvants (e.g., aluminum hydroxide); and preservatives; sterile diluents such as water for injection, saline solution, preferably physiological saline, Ringer's solution, isotonic sodium chloride, fixed oils such as synthetic mono- or diglycerides, which may serve as the solvent or suspending medium, polyethylene glycols, glycerin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. The parenteral preparation can be enclosed in ampoules, disposable syringes or multiple dose vials made of glass or plastic.

### Target Protein (e.g., Biomolecule of Interest)

The terms “polypeptide” or “protein” are used interchangeably throughout the disclosure and refer to a molecule comprising two or more amino acid residues joined by peptide bonds. Polypeptides and proteins also include macromolecules having one or more deletions from, insertions to, and/or substitutions of the amino acid residues of the native sequence, that is, a polypeptide or protein produced by a naturally occurring and non-recombinant cell; or is produced by a genetically engineered or recombinant cell, and comprise molecules having one or more deletions from, insertions to, and/or substitutions of the amino acid residues of the amino acid sequence of the native protein. Polypeptides and proteins also include amino acid polymers in which one or more amino acids are chemical analogs of a corresponding naturally occurring amino acid and polymers. Polypeptides and proteins are also inclusive of modifications including, but not limited to, derivatizations such as glycosylation, lipid attachment, sulfation, gamma-carboxylation of glutamic acid residues, hydroxylation and ADP-ribosylation.

The terms “polynucleotide”, “nucleic acid molecule”, or “engineered nucleic acid molecule” are used interchangeably throughout the disclosure and include both single-stranded and double-stranded nucleic acids, including genomic DNA, RNA, mRNA, cDNA, nucleic acids of synthetic origin, or some combination thereof which is not associated with sequences normally found in nature. The terms “isolated polynucleotide”, “isolated nucleic acid molecule” or “isolated engineered nucleic acid molecule” specifically refer to sequences not normally found in nature, such as sequences of synthetic origin. Isolated nucleic acid molecules comprising specified sequences may include, in addition to the specified sequences, coding sequences for up to ten or even up to twenty other proteins or portions thereof or may include operably linked regulatory sequences that control expression of the coding region of the recited nucleic acid sequences, and/or may include vector sequences. The nucleotides comprising the nucleic acid molecules can be ribonucleotides or deoxyribonucleotides or a modified form of either type of nucleotide. The modifications include base modifications such as bromouridine and inosine derivatives, ribose modifications such as 2′,3′-dideoxyribose, and internucleotide linkage modifications such as phosphorothioate, phosphorodithioate, phosphoroselenoate, phosphorodiselenoate, phosphoroanilothioate, phosphoraniladate and phosphoroamidate.

Biomolecules (e.g., polypeptides and proteins) of interest can be of scientific or commercial interest, including protein-based therapeutics. Biomolecules (e.g., proteins) of interest include, among other things, secreted proteins, non-secreted proteins, intracellular proteins or membrane-bound proteins. Biomolecules of interest can be produced by recombinant animal cell lines using cell culture methods and may be referred to as “recombinant proteins” to indicate the source rather than the structure of the protein biomolecules. Proteins of the disclosure may have the same structure as a naturally occurring protein, may be a fragment thereof such as a fragment specifically binding to a binding partner or an enzymatically active fragment, and the protein may also be a chimera or fusion of at least parts of two or more proteins. The expressed protein(s) may be produced intracellularly or secreted into the culture medium from which it can be recovered and/or collected according to the methods of the disclosure. The term “isolated protein” or “isolated recombinant protein” refers to a polypeptide or protein of interest, that is purified away from proteins or polypeptides or other contaminants that would interfere with its therapeutic, diagnostic, prophylactic, research or other use. Biomolecules of interest include proteins that exert a therapeutic effect by binding a target, particularly a target among those listed below, including targets derived therefrom, targets related thereto, and modifications thereof.

By “purifying” is meant increasing the degree of purity of the protein in the composition by removing (partially or completely) at least one impurity from the composition. Recovery and purification of proteins is accomplished by the methods of the disclosure, resulting in a more “homogeneous” protein composition that meets yield and product quality benchmarks (such as reduced product-related impurities and increased product quality).

As used herein, the term “isolated” means (i) free of at least some other proteins or polynucleotides with which it would normally be found, (ii) is essentially free of other proteins or polynucleotides from the same source, e.g., from the same species or from the same cell, (iii) separated from at least about 50 percent of polypeptides, polynucleotides, lipids, carbohydrates, or other materials with which it is associated in nature, (iv) operably associated (by covalent or noncovalent interaction) with a polypeptide or polynucleotide with which it is not associated in nature, or (v) does not occur in nature.

Biomolecules (e.g., proteins) of interest include “antigen-binding proteins”. Antigen-binding protein refers to proteins, polypeptides, or fragments thereof that comprise an antigen-binding region or antigen-binding portion that has affinity for another molecule to which it binds (antigen). Antigen-binding proteins encompass antibodies, peptibodies, antibody fragments, antibody derivatives, antibody analogs, fusion proteins (including single-chain variable fragments (scFvs) and double-chain (divalent) scFvs), muteins, multispecific proteins, and bispecific proteins.

An scFv is a single-chain antibody fragment having the variable regions of the heavy and light chains of an antibody linked together. See U.S. Pat. Nos. 7,741,465, and 6,319,494 as well as Eshhar et al., Cancer Immunol Immunotherapy (1997) 45: 131-136, all incorporated herein by reference in relevant parts. An scFv retains the parent antibody's ability to interact specifically with target antigen.

The term “antibody” includes reference to both glycosylated and non-glycosylated immunoglobulins of any isotype or subclass or to an antigen-binding region thereof that competes with the intact antibody for specific binding. Unless otherwise specified, antibodies include human, humanized, chimeric, multi-specific, monoclonal, polyclonal, heteroIgG, bispecific, and oligomers or antigen-binding fragments thereof. Antibodies include the IgG1-, IgG2-, IgG3- or IgG4-type. Also included are proteins having an antigen binding fragment or region such as Fab, Fab′, F(ab′)2, Fv, diabodies, Fd, dAb, maxibodies, single-chain antibody molecules, single-domain V_{H}H, complementarity determining region (CDR) fragments, scFv, diabodies, triabodies, tetrabodies and polypeptides that contain at least a portion of an immunoglobulin that is sufficient to confer specific antigen binding to a target polypeptide.

Also included are human, humanized, and other antigen-binding proteins, such as human and humanized antibodies, that do not engender significantly deleterious immune responses when administered to a human.

Modified proteins are also included, such as proteins modified chemically by a non-covalent bond, covalent bond, or both a covalent and non-covalent bond. Also included are proteins further comprising one or more post-translational modifications that may be made by cellular modification systems or modifications introduced ex vivo by enzymatic and/or chemical methods or introduced in any other way known in the art.

“Multispecific constructs”, “multispecific protein”, and “multispecific antibody” are used herein to refer to proteins that are recombinantly engineered to simultaneously bind and neutralize at least two different antigens or at least two different epitopes on the same antigen. For example, multispecific proteins may be engineered to target immune effectors in combination with targeting cytotoxic agents to tumors or infectious agents. Multispecific proteins include trispecific antibodies, tetravalent bispecific antibodies, multispecific proteins without antibody components such as dia-, tria- or tetrabodies, minibodies, and single-chain proteins capable of binding multiple targets. Coloma, M. J., et al., Nature Biotech. 15:159-163 (1997), incorporated herein by reference in relevant part.

The most common and most diverse group of multispecific proteins are those that bind two antigens, referred to herein as “bispecific”, “bispecific constructs”, “bispecific proteins”, and “bispecific antibodies”. Bispecific proteins can be grouped in two broad categories: immunoglobulin G (IgG)-like molecules and non-IgG-like molecules. IgG-like molecules retain Fc-mediated effector functions, such as antibody-dependent cell mediated cytotoxicity (ADCC), complement-dependent cytotoxicity (CDC), and antibody-dependent cellular phagocytosis (ADCP). The Fc region helps improve solubility and stability and facilitates some purification operations. Non-IgG-like molecules are smaller, enhancing tissue penetration (see Sedykh et al., Drug Design, Development and Therapy 18(12), 195-208, 2018; Fan et al., J Hematol & Oncology 8:130-143, 2015; Spiess et al., Mol Immunol 67, 95-106, 2015; Williams et al., Chapter 41 Process Design for Bispecific Antibodies in Biopharmaceutical Processing Development, Design and Implementation of Manufacturing Processes, Jagschies et al., eds., 2018, pages 837-855, all incorporated herein by reference in relevant parts). Bispecific proteins are sometimes used as frameworks for additional components having binding specificities to different antigens or numbers of epitopes, increasing the binding specificity of the molecule.

The formats for bispecific proteins, which include bispecific antibodies, are constantly evolving and include, but are not limited to, single-chain antibodies, quadromas, knobs-in-holes, cross-mAbs, dual variable domain IgG (DVD-IgG), IgG single-chain Fv (scFv), scFv-CH3 KIH, dual action Fab (DAF), half-molecule exchange, KA-bodies, tandem scFv, scFv-Fc, diabodies, single-chain diabodies (scDiabodies), scDiabodies-CH3, triple body, miniantibody, minibody, TriBi minibody, tandem diabodies, scDiabody-HAS, Tandem scFv-toxin, dual-affinity retargeting molecules (DARTs), nanobody, nanobody-HSA, dock and lock (DNL), strand exchange engineered domain SEEDbody, Triomab, leucine zipper (LUZ-Y), antibodies made using XmAb® technology; Fab-arm exchange, DutaMab, DT-IgG, charged pair, Fcab, orthogonal Fab, IgG(H)-scFv, scFV-(H)IgG, IgG(L)-scFV, IgG(L1H1)-Fv, IgG(H)-V, V(H)-IgG, IgG(L)-V V(L)-IgG, KIH IgG-scFab, 2scFV-IgG, IgG-2scFv, scFv4-Ig, Zybody, DVI-Ig4 (four-in-one), Fab-scFv, scFv-CH-CL-scFV, F(ab′)2-scFv2, scFv-KIH, Fab-scFv-Fc, tetravalent HCAb, scDiabody-Fc, diabody-Fc, intrabody, ImmTAC, HSABody, IgG-IgG, Cov-X-Body, scFv1-PEG-scFv2, bi-specific T cell engagers including BiTE® molecules (Fan supra; Spiess supra; Sedykh supra; Seimetz et al., Cancer Treat Rev 36(6) 458-67, 2010; Shulka and Norman, Chapter 26 Downstream Processing of Fc Fusion Proteins, Bispecific Antibodies, and Antibody-Drug Conjugates, in Process Scale Purification of Antibodies Second Edition, Uwe Gottschalk editor, p559-594, John Wiley & Sons, 2017; Moore et al., MAbs 3:6, 546-557, 2011, each incorporated herein by reference in relevant part). Biomolecules (e.g., proteins) of interest may also include recombinant fusion proteins comprising, for example, a multimerization domain, such as a leucine zipper, a coiled coil, an Fc portion of an immunoglobulin, and the like. Also included are proteins comprising all or part of the amino acid sequences of differentiation antigens (referred to as CD proteins) or their ligands, or proteins substantially similar to either of these.

Biomolecules (e.g., proteins) of interest also include genetically engineered receptors such as chimeric antigen receptors (CARs or CAR-Ts) and T cell receptors (TCRs), as well as other proteins comprising an antigen-binding molecule that interacts with the targeted antigen. CARs can be engineered to bind to an antigen (such as a cell-surface antigen) by incorporating an antigen-binding molecule that interacts with that targeted antigen. CARs typically incorporate an antigen binding domain (such as scFv) in tandem with one or more co-stimulatory (“signaling”) domains and one or more intracellular activating domains.

In some embodiments, biomolecules (e.g., proteins) of interest may include colony-stimulating factors, such as granulocyte colony-stimulating factor (G-CSF). Such G-CSF agents include, but are not limited to, Neupogen® (filgrastim) and Neulasta® (pegfilgrastim). Also included are erythropoiesis-stimulating agents (ESA), such as Epogena® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin zeta, epoetin theta, epoetin delta, epoetin omega, epoetin iota, tissue plasminogen activator, GLP-1 receptor agonists, as well as the molecules or variants or analogs thereof and biosimilars of any of the foregoing.

In some embodiments, biomolecules (e.g., proteins) of interest may include proteins that bind specifically to one or more CD proteins, HER receptor family proteins, cell adhesion molecules, growth factors, nerve growth factors, fibroblast growth factors, transforming growth factors (TGF), insulin-like growth factors, osteoinductive factors, insulin and insulin-related proteins, coagulation and coagulation-related proteins, colony stimulating factors (CSFs), other blood and serum proteins, blood group antigens, receptors, receptor-associated proteins, growth hormones, growth hormone receptors, T-cell receptors, neurotrophic factors, neurotrophins, relaxins, interferons, interleukins, viral antigens, lipoproteins, integrins, rheumatoid factors, immunotoxins, surface-membrane proteins, transport proteins, homing receptors, addressins, regulatory proteins, and immunoadhesins.

In some embodiments, biomolecules (e.g., proteins) of interest bind to one of more of the following, alone or in any combination: CD proteins including but not limited to CD3, CD4, CD5, CD7, CD8, CD19, CD20, CD22, CD25, CD30, CD33, CD34, CD38, CD40, CD70, CD123, CD133, CD138, CD171, and CD174, HER receptor family proteins, including, for instance, HER2, HER3, HER4, and the EGF receptor, EGFRvIII, cell adhesion molecules, for example, LFA-1, Mol, p150,95, VLA-4, ICAM-1, VCAM, and alpha v/beta 3 integrin, growth factors, including but not limited to, vascular endothelial growth factor (“VEGF”); VEGFR2, growth hormone, thyroid-stimulating hormone, follicle-stimulating hormone, luteinizing hormone, growth hormone releasing factor, parathyroid hormone, mullerian-inhibiting substance, human macrophage inflammatory protein (MIP-1-alpha), erythropoietin (EPO), nerve growth factor, such as NGF-beta, platelet-derived growth factor (PDGF), fibroblast growth factors including, for instance, aFGF and bFGF, epidermal growth factor (EGF), Cripto, transforming growth factors (TGFs) including, among others, TGF-α and TGF-β, including TGF-β1, TGF-β2, TGF-β3, TGF-β4, and TGF-β5, insulin-like growth factors-I and -II (IGF-I and IGF-II), des(1-3)-IGF-I (brain IGF-I), and osteoinductive factors, insulins and insulin-related proteins including, but not limited to, insulin, insulin A-chain, insulin B-chain, proinsulin, and insulin-like growth factor binding proteins; (coagulation and coagulation-related proteins, such as factor VIII, tissue factor, von Willebrand factor, protein C, alpha-1-antitrypsin, plasminogen activators, such as urokinase and tissue plasminogen activator (“t-PA”), bombazine, thrombin, thrombopoietin, and thrombopoietin receptor, colony stimulating factors (CSFs) including the following, among others, M-CSF, GM-CSF, and G-CSF, other blood and serum proteins including, but not limited to, albumin, IgE, and blood group antigens, receptors and receptor-associated proteins including, for example, flk2/flt3 receptor, obesity (OB) receptor, growth hormone receptors, T-cell receptors, neurotrophic factors including, but not limited to, bone-derived neurotrophic factor (BDNF) and neurotrophin-3, -4, -5, or -6 (NT-3, NT-4, NT-5, or NT-6), relaxin A-chain, relaxin B-chain, and prorelaxin, interferons including, for example, interferon-alpha, -beta, and -gamma, interleukins (ILs) and their receptors, e.g., IL-1 to IL-10, IL-12, IL-15, IL-17, IL-23, IL-12/IL-23, IL-2Ra, IL1-R1, IL-6 receptor, IL-4 receptor and/or IL-13 receptor, IL-13RA2, or IL-17 receptor, IL-1 RAP, IL1-α, IL-1β, viral antigens including, but not limited to, an AIDS envelope viral antigen, lipoproteins, calcitonin, glucagon, atrial natriuretic factor, lung surfactant, tumor necrosis factor-alpha and -beta, enkephalinase, BCMA, IgKappa, ROR-1, ERBB2, mesothelin, RANTES (regulated on activation normally T-cell expressed and secreted), mouse gonadotropin-associated peptide, DNase, FR-alpha, inhibin, and activin, integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, MIC (MIC-a, MIC-B), ULBP 1-6, EPCAM, PSA, addressins, regulatory proteins, immunoadhesins, antigen-binding proteins, somatotropin, CTGF, CTLA4, eotaxin-1, MUC1, CEA, c-MET, Claudin-18, GPC-3, EPHA2, FPA, LMP1, MG7, NY-ESO-1, PSCA, ganglioside GD2, ganglioside GM2, BAFF, OPGL (RANKL), myostatin, Dickkopf-1 (DKK-1), Ang2, NGF, IGF-1 receptor, hepatocyte growth factor (HGF), TRAIL-R2, c-Kit, B7RP-1, PSMA, P-cadherin, NKG2D-1, programmed cell death protein 1 and ligand PD1 and PDL1, mannose receptor/hCGβ, hepatitis-C virus, mesothelin, SS1(dsFv)PE38 conjugate, Legionella pneumophila (Ily), gpA33, B7H3, interferon (IFN) gamma, interferon gamma-induced protein 10 (IP10), IFNAR, TALL-1, thymic stromal lymphopoietin (TSLP), proprotein convertase subtilisin/Kexin Type 9 (PCSK9), stem cell factors, Flt-3, calcitonin gene-related peptide (CGRP), OX40L, α4β7 integrin, platelet specific (platelet glycoprotein IIb/IIIb (PAC-1), transforming growth factor beta (TFGβ), Zona pellucida sperm-binding protein 3 (ZP-3), TWEAK, platelet-derived growth factor receptor alpha (PDGFRα), sclerostin, and biologically active fragments or variants of any of the foregoing.

In some embodiments, biomolecules (e.g., proteins) of interest include abciximab, adalimumab, adecatumumab, aflibercept, alemtuzumab, alirocumab, anakinra, atacicept, basiliximab, belimumab, bevacizumab, biosozumab, brentuximab vedotin, brodalumab, cantuzumab mertansine, canakinumab, cetuximab, certolizumab pegol, conatumumab, daclizumab, denosumab, eculizumab, edrecolomab, efalizumab, epratuzumab, etanercept, evolocumab, galiximab, ganitumab, gemtuzumab, golimumab, ibritumomab tiuxetan, infliximab, ipilimumab, lerdelimumab, lumiliximab, lxdkizumab, mapatumumab, motesanib diphosphate, muromonab-CD3, natalizumab, nesiritide, nimotuzumab, nivolumab, ocrelizumab, ofatumumab, omalizumab, oprelvekin, palivizumab, panitumumab, pembrolizumab, pertuzumab, pexelizumab, ranibizumab, rilotumumab, rituximab, romiplostim, romosozumab, sargamostim, tocilizumab, tositumomab, trastuzumab, ustekinumab, vedolizumab, visilizumab, volociximab, zanolimumab, zalutumumab, and biosimilars of any of the foregoing.

In some embodiments, biomolecules (e.g., proteins) of interest may include blinatumomab, catumaxomab, ertumaxomab, solitomab, targomiRs, lutikizumab (ABT981), vanucizumab (RG7221), remtolumab (ABT122), ozoralixumab (ATN103), floteuzmab (MGD006), pasotuxizumab (AMG112, MT112), lymphomun (FBTA05), (ATN-103), AMG211 (MT111, Medi-1565), AMG330, AMG420 (B1836909), AMG-110 (MT110), MDX-447, TF2, rM28, HER2Bi-aATC, GD2Bi-aATC, MGD006, MGD007, MGD009, MGD010, MGD011 (JNJ64052781), IMCgp100, indium-labeled IMP-205, xm734, LY3164530, OMP-305BB3, REGN1979, COV322, ABT112, ABT165, RG-6013 (ACE910), RG7597 (MEDH7945A), RG7802, RG7813(RO6895882), RG7386, BITS7201A (RG7990), RG7716, BFKF8488A (RG7992), MCLA-128, MM-111, MM141, MOR209/ES414, MSB0010841, ALX-0061, ALX0761, ALX0141; B11034020, AFM13, AFM11, SAR156597, FBTA05, PF06671008, GSK2434735, MED13902, MED10700, MED17352, as well as modified molecules or variants or analogs thereof and biosimilars of any of the foregoing.

Biomolecules (e.g., proteins) of interest according to the disclosure encompass all of the foregoing and further include antibodies comprising 1, 2, 3, 4, 5, or 6 of the complementarity determining regions (CDRs) of any of the aforementioned antibodies. Also included are variants that comprise a region that is 70% or more, 80% or more, 90% or more, 95% or more, 97% or more, 98% or more, or 99% or more identical in amino acid sequence to a reference amino acid sequence of a biomolecule of interest in the form of a protein. Identity in this regard can be determined using a variety of well-known and readily available forms of amino acid sequence analysis software. Preferred software includes those that implement the Smith-Waterman algorithms, considered a satisfactory solution to the problem of searching and aligning sequences. Other algorithms also may be employed, particularly where speed is an important consideration. Commonly employed programs for alignment and homology matching of DNAs, RNAs, and polypeptides that can be used in this regard include FASTA, TFASTA, BLASTN, BLASTP, BLASTX, TBLASTN, PROSRCH, BLAZE, and MPSRCH, the latter being an implementation of the Smith-Waterman algorithm for execution on massively parallel processors made by MasPar.

Chimeric antigen receptors (CARs) incorporate one or more costimulatory (signaling) domains known in the art to increase the potency of the CAR-T cell response. See U.S. Pat. Nos. 7,741,465, and 6,319,494, as well as Krause et al. and Finney et al. (supra), Song et al., Blood 119:696-706 (2012); Kalos et al., Sci Transl. Med. 3:95 (2011); Porter et al., N. Engl. J. Med. 365:725-33 (2011), and Gross et al., Annu. Rev. Pharmacol. Toxicol. 56:59-83 (2016), each incorporated herein by reference in relevant part. Exemplary costimulatory domains for incorporation into CARs can be derived from, among other sources, CD28, CD28T, OX40, 4-1BB/CD137, CD2, CD3 (alpha, beta, delta, epsilon, gamma, zeta), CD4, CD5, CD7, CD8, CD9, CD16, CD22, CD27, CD30, CD 33, CD37, CD40, CD 45, CD64, CD80, CD86, CD134, CD137, CD154, PD-1, ICOS, lymphocyte function-associated antigen-1 (LFA-1 (CDI Ia/CD18), CD247, CD276 (B7-H3), LIGHT (tumor necrosis factor superfamily member 14; TNFSF14), NKG2C, Ig alpha (CD79a), DAP-10, Fc gamma receptor, MHC class I molecule, TNF, TNFr, integrin, signaling lymphocytic activation molecule, BTLA, Toll ligand receptor, ICAM-1, B7-H3, CDS, ICAM-1, GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL-2R beta, IL-2R gamma, IL-7R alpha, ITGA4, VLA1, CD49a, ITGA4, IA4, CD49D, ITGA6, VLA-6, CD49f, ITGAD, CDI-Id, ITGAE, CD103, ITGAL, CDI-Ia, LFA-1, ITGAM, CDI-Ib, ITGAX, CDI-Ic, ITGBI, CD29, ITGB2, CD18, LFA-1, ITGB7, NKG2D, TNFR2, TRANCE/RANKL, DNAM1 (CD226), SLAMF4 (CD244, 2B4), CD84, CD96 (Tactile), CEACAM1, CRT AM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, 41-BB, GADS, SLP-76, PAG/Cbp, CD19a, CD83 ligand, or fragments or combinations thereof. The costimulatory domain can comprise one or more of an extracellular portion, a transmembrane portion, and an intracellular portion of the protein from which the costimulatory domain is derived.

The following examples, both actual and prophetic, are provided for the purpose of illustrating specific embodiments or features of the present invention and are not intended to limit its scope.

### EXAMPLES

### Example 1 Single-Use Continuous Solids Discharge Disc Stack Centrifugation, Flocculation and Depth Filtration of High Density Cell Cultures

The example provides a harvest method for purifying a target protein from a high-density continuous perfusion mammalian cell culture using a purification protocol that makes use of a single-use continuous solids discharge disc stack centrifugation step followed by flocculation, and depth filtration. The harvest method results in a high-yield, purified protein from high-density cultures using an unconventional yet effective process.

Nine different perfusion cultures were executed at 500 L scale. The cultures comprised one of four monoclonal antibodies (mAb), i.e., mAb 1 (Runs 1, 2, and 3), mAb 2, mAb 3, or mAb 4), xmAbs (Runs 1 and 2) or the cultures comprised one of two bispecific antibodies (Bsp), i.e., Bsp 1 and Bsp 2.

CHO cells expressing the monoclonal or bispecific antibodies were inoculated into 500 L single-use bioreactors at a working volume of 450-500 L of a serum-free, chemically defined, batch medium on day 0. The cultures were initiated in batch mode at 36° C., with agitation. The cultures were then continuously perfused using an X-CELL ATF-6® alternating tangential flow (ATF) filtration system (Repligen, Waltham, MA) with an ATF-6 filter with a nominal surface area of 2.1 m² and a serum-free, chemically defined perfusion medium until harvest. The cultures were subjected to a low-temperature shift (32-33° C.) to increase production and maintained at the lower temperature until harvest.

On day 15 or day 18 the contents of the bioreactors were bulk-harvested. Initially, the culture was cooled to 10° C. The cell culture broth was then harvested directly at 10° C. or collected into a holding tank and held at 4° C. The harvest cell viability, viable cell density (VCD), percent packed cell volume (% PCV) and baseline turbidity of the cultures were determined using conventional techniques, and the results are presented in Table 1.

Undiluted cell culture was subjected to continuous centrifugation using a 500 L single-use continuous solids discharge disc stack centrifuge (Alfa Laval Primo™, Fresno, CA). The cell culture was continuously loaded into the centrifuge by an in-line mechanism at a specific flow rate (Table 1). The centrifuge was operated at 5.25 E-9 m/s to 1.5×E-8 m/s equivalent sedimentation rate (Q/Sigma) or 0.6 to 1.8 (L/h/KQ) with % PCV values of 16-30%.

The feed flow rate and heavy-phase pump flow rate were modulated to maintain a desired heavy-phase % PCV. The bowl speed and feed flow rate were controlled to maintain a desired light-phase turbidity value. The reported values for centrifuge feed flow rate, centrate flow rate, heavy-phase flow rate, and harvest yield are average values of different sample time points over the course of the centrifugation process for a given sample (see Table 1).

As shown in Table 1, the average bowl speed varied depending on the molecule being harvested and was between 4713-5200 rpm using the Alfa Laval Primo™ bowl. The average centrifuge feed flow rate was between 0.7-2.0 L/min (LPM). The centrate was continuously collected into a harvest tank and held at 10° C. with mixing. The concentrate (heavy phase) was discharged to waste. The average centrate flow rate was between 0.79-1.51 L/min. The average concentrate (heavy phase) flow rate was between 0.29-0.61 L/min. The average concentrate (heavy phase) % PCV was between 77-96%. In-line centrate measurements were taken periodically, and the average centrate turbidity was between 165-940 Nephelometric Turbidity Units (NTUs). The average theoretical centrifugation yield was between 88-99%. In performing single-use continuous solids discharge disc stack centrifugation according to the methods of the disclosure, attention is paid to the feed flow rate, bowl speed and the heavy-phase % PCV target, which is influenced by the cell density of the feed and the flow rate of the heavy-phase pump. These parameters are summarized using Q/Sigma values, as noted herein, and Q/Sigma values are positively correlated with turbidity because an increase in the feed flow rate (Q) for a given equivalent settling area (Sigma) will increase turbidity. Important centrifugation operation parameters include the centrifuge bowl speed, the feed flow rate, the cell culture solid content (PCV), and the heavy-phase (cell paste) solid content (PCV). Exemplary values for these parameters are disclosed throughout the application. These parameters are adjusted to control the normalized sedimentation rate (Q/Sigma) and theoretical step yield. As is known in the art, Q/Sigma provides a measure of the normalized sedimentation rate, with Q being the feed flow rate and Sigma being the normalized settling area of the spinning bowl. To achieve a relatively lower Q/Sigma, the centrifuge is operated at a lower feed flow rate to allow more time in residence for cells and larger debris to be captured, resulting in less debris in the centrate.

Centrate turbidity is generally proportional to particle, especially fine particle, concentration. As the data herein show, harvest and centrate turbidity vary to a degree due to differences in cell culture, cell density, packed cell volume and percent viability of various cultures subject to harvest according to the disclosure. It is noteworthy that existing technologies using intermittent solids discharge centrifugation are suitable only for lower cell density cultures with PCV values of 3-12% at cell culture harvest. Other approaches to obtaining proteins of interest from cell culture using fed-batch cell culturing result in lower turbidity, but that is due to lower cell density and a lower % PCV, consistent with a reduced particle burden. In some approaches, a harvest operation is engineered in which a cell culture with a relatively low % PCV is mixed with cell paste. The result is a mixed cell-containing fluid in which the % PCV is artificially increased in an environment with artificially high cell viability and correspondingly low cell debris, allowing for high cell culture feed or flow rates that would not be suitable for real-world cell culture harvest operations.

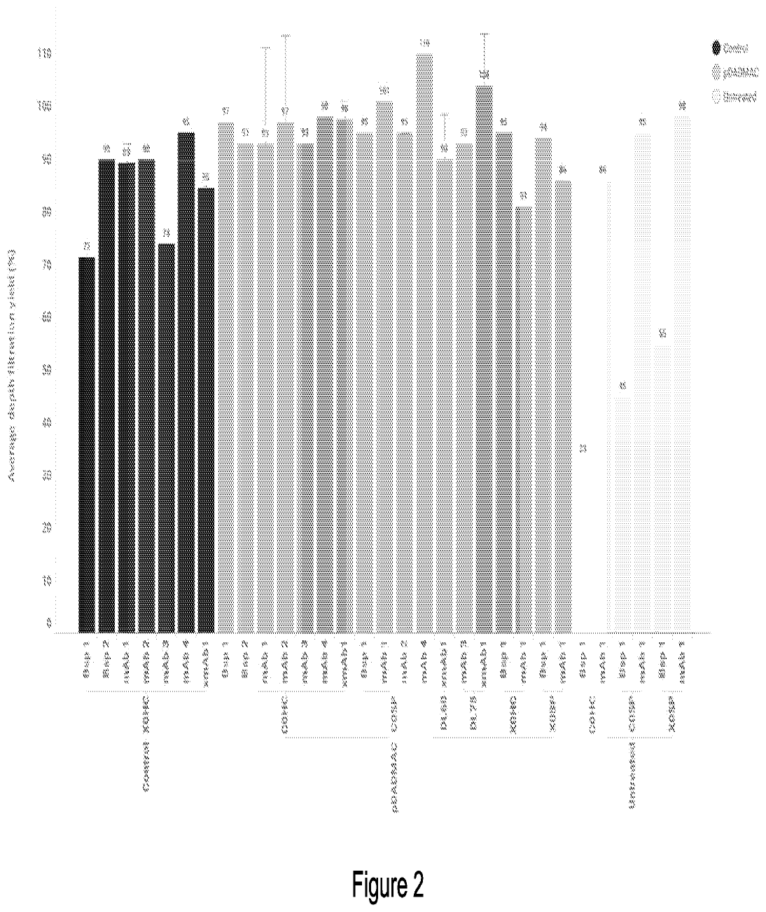

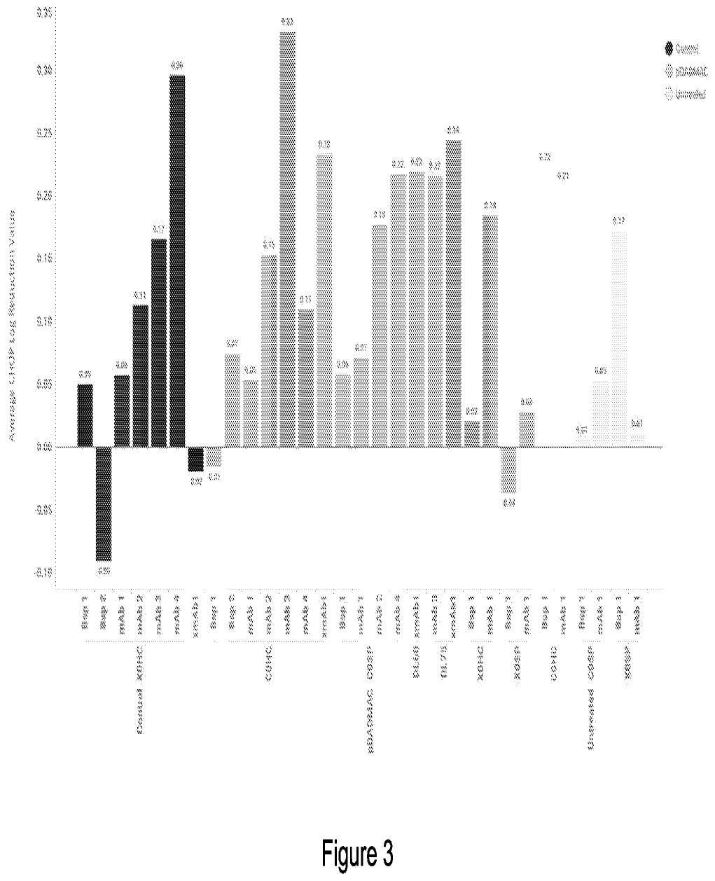

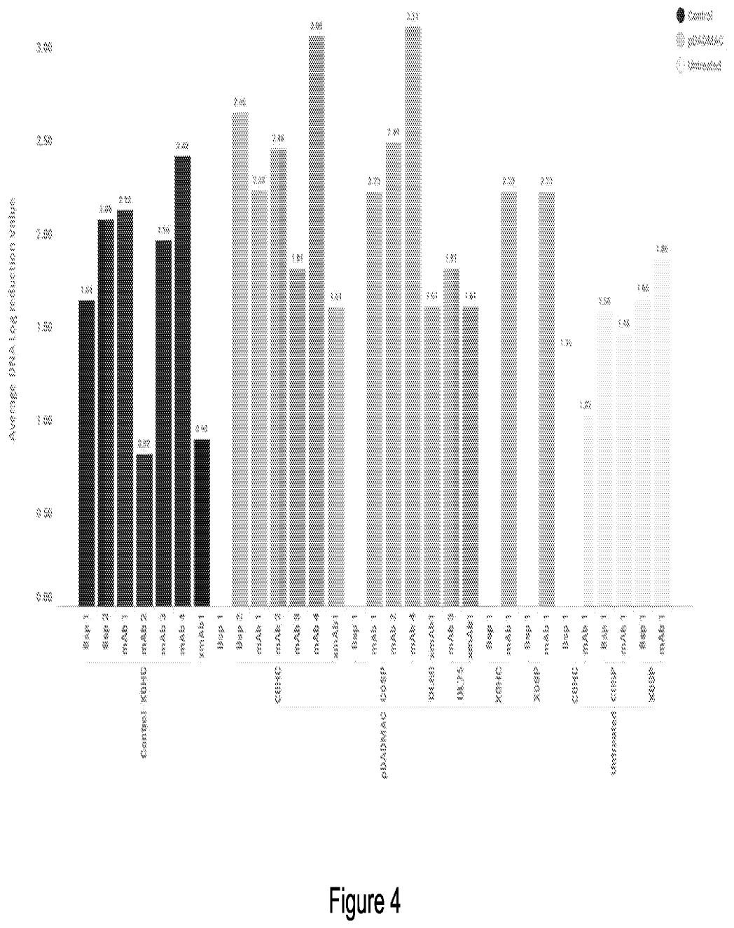

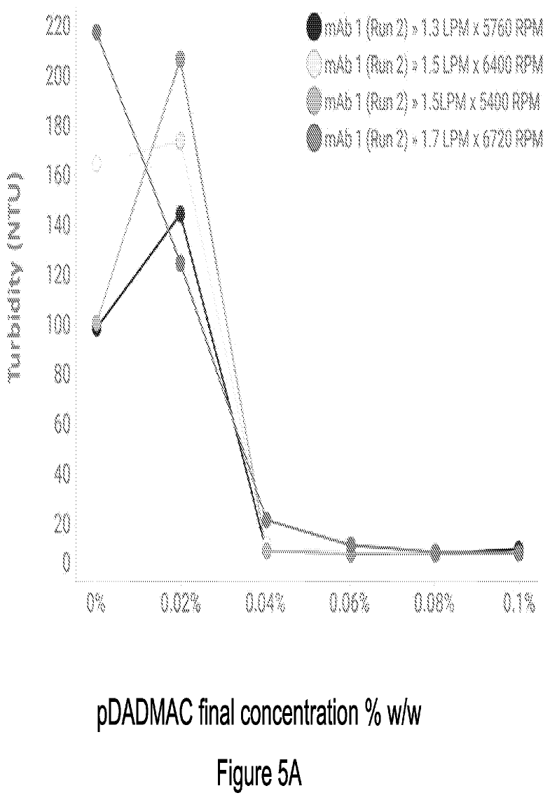

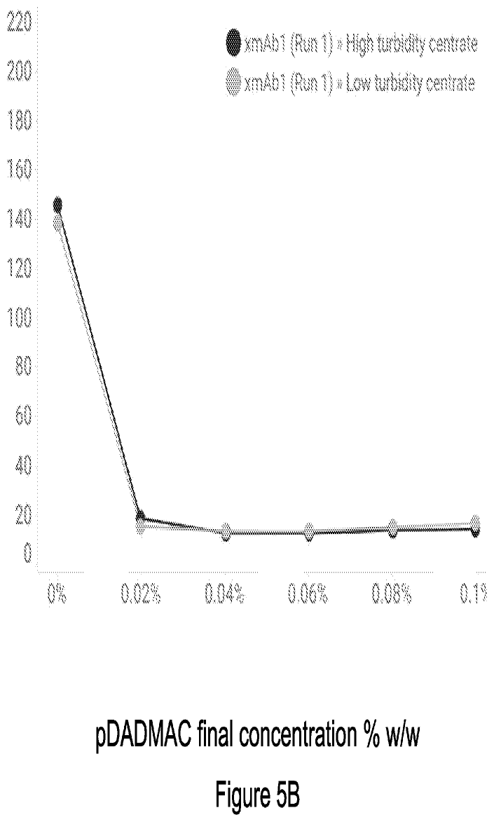

As the experiments disclosed herein establish, single-use continuous solids discharge disc stack centrifugation was effective in removing whole cells from cell culture broth with high yield. The average centrate turbidity observed was 165 to 941 NTU, however, indicating that a high level of cell debris fragments remained in solution. A major contributor to turbidity in centrate fluids is accumulated cell debris, which primarily results from cell death mechanisms in the bioreactor or cell culture vessel but can also be affected by how the centrifuge is operated. A lower feed flow rate and a higher bowl speed will decrease turbidity, for example. Variation in turbidity complicates depth filter sizing. This complication was overcome by adding a post-centrifuge flocculation step. Adding the flocculation step improves process robustness against the variations in turbidity in real-world target protein harvests. The benefit of adding a flocculation step was revealed by a comparative study in which centrate clarification by flocculation followed by depth filtration, was compared to clarification by depth filtration alone (see Example 2). The effect of flocculation concentration on turbidity was first studied by adding pDADMAC at varying concentrations to centrate followed by lab-scale centrifugation and supernatant turbidity measurements. Low turbidity was observed at concentrations of 0.04% to 0.10% (w/w) pDADMAC (FIG. 5). A concentration of 0.05% (w/v) pDADMAC followed by depth filtration was tested using different depth filters and the results were compared to the results obtained using depth filtration alone. More particularly, results from these experiments showed a 0.85- to 16.2-fold increase in depth filtration load capacity following pDADMAC treatment when compared to depth filtration alone (FIG. 1). In addition to increased depth filter loading level, similar or improved DNA clearance and host cell protein (HCP) clearance were observed with flocculation and depth filtration in comparison to depth filtration alone (FIGS. 3 and 4). Improved filter loading and impurity clearance did not negatively impact step yield when compared to depth filtration alone (FIG. 2). In general, similar or improved step yields were observed for flocculation and depth filtration when compared to depth alone. Levels of product loss are acceptable in view of the increased clarity of the centrate fluids, which were suitable for direct application to chromatographic fractionations, e.g., affinity binding of antibody biologics to Protein-A columns.

TABLE 1
Single-use continuous solids discharge disc stack centrifuge parameters for various large-scale (500 L) runs

| | | | | | | Avg. | Avg. | | | Avg. | | | |
|---|---|---|---|---|---|---|---|---|---|---|---|---|---|
| | Culture | | Harvest | Harvest | | Angular | Centrifuge | | Avg. | Heavy | Avg. | Avg. | |
| | Dura- | Harvest | Sample | VCD | Harvest | Velocity | Feed Flow | | Centrate | Phase | Heavy | Centrate | [symbol] |
| Sample | tion | Day | Turbidity | (×10⁵ | Viability | of Bowl | Rate | Q/Sigma | Flow | Flow Rate | phase % | Turbidity | Cen [symbol] |
| Name | (Days) | % PCV | (NTU) | cells/ml) | (%) | (RPM) | (LPM) | (m/s) | (LPM) | (LPM) | PCV | (NTU) | Yi [symbol] |
| mAb 1 | 18 | 24 | 250 | 218 | 88 | 5200 | 2.0 | 1.50E−08 | 1.51 | 0.61 | 84 | 272 | [symbol] |
| (Run 1) | | | | | | | | | | | | | |
| mAb 1 | 18 | 24 | 250 | 218 | 79 | 5200 | 1.5 | 1.12E−08 | 1.5 | 0.45 | 82 | 182 | [symbol] |
| (Run 2) | | | | | | | | | | | | | |
| mAb 2 | 15 | 16 | 184 | 327 | 81 | 5200 | 1.5 | 1.12E−08 | 1.19 | 0.3 | 94 | 322 | [symbol] |
| mAb 3 | 15 | 27 | 524 | 316 | n/a | 5147 | 1.5 | 1.12E−08 | 0.94 | 0.54 | 83 | 669 | [symbol] |
| xmAb 1 | 15 | 26 | 404 | 542 | 88 | 5140 | 1.3 | 9.74E−09 | 0.86 | 0.45 | 93 | 299 | [symbol] |
| (Run 1) | | | | | | | | | | | | | |
| xmAb 1 | 15 | 25 | 300 | 641 | 89 | 5138 | 1.2 | 8.99E−09 | 0.79 | 0.41 | 84 | 235 | [symbol] |
| (Run 2) | | | | | | | | | | | | | |
| Bispe- | 15 | 30 | N/A | 602 | 88 | 5200 | 0.7 | 5.25E−09 | 1.09 | 0.5 | 77 | 165 | [symbol] |
| cific 1 | | | | | | | | | | | | | |
| mAb 4 | 15 | 23 | N/A | 526 | 88 | 4713 | 1.7 | 1.27E−08 | 1.28 | 0.45 | 96 | 941 | [symbol] |
| Bispe- | 15 | 18 | 195 | 208 | 44 | 5133 | 1.3 | 9.74E−09 | 1.02 | 0.29 | 82 | n/a | [symbol] |
| cific 2 | | | | | | | | | | | | | |

Avg—Average
NTU—Nephelometric turbidity units
VCD—Viable cell density
LPM—Liters per minute [symbol]
indicates data missing or illegible when filed

### Example 2 Depth Filtration of Flocculated Centrate

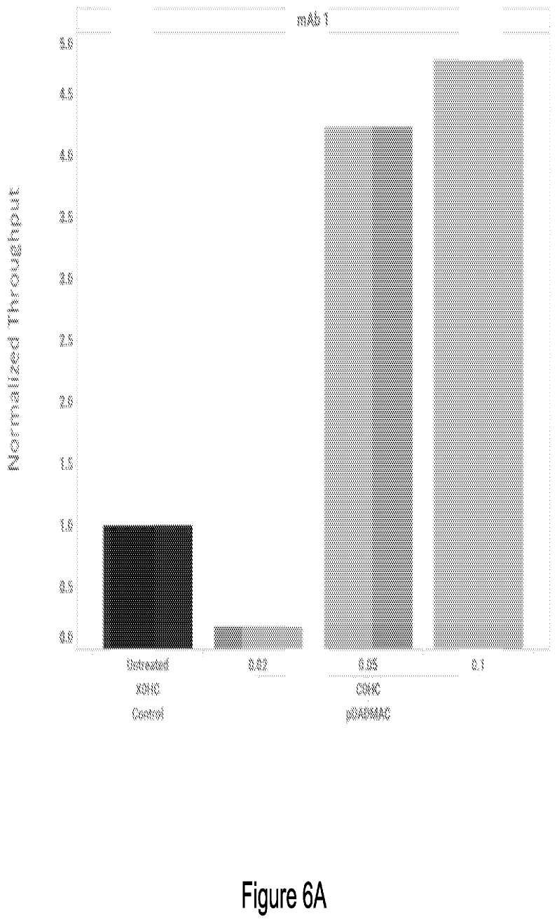

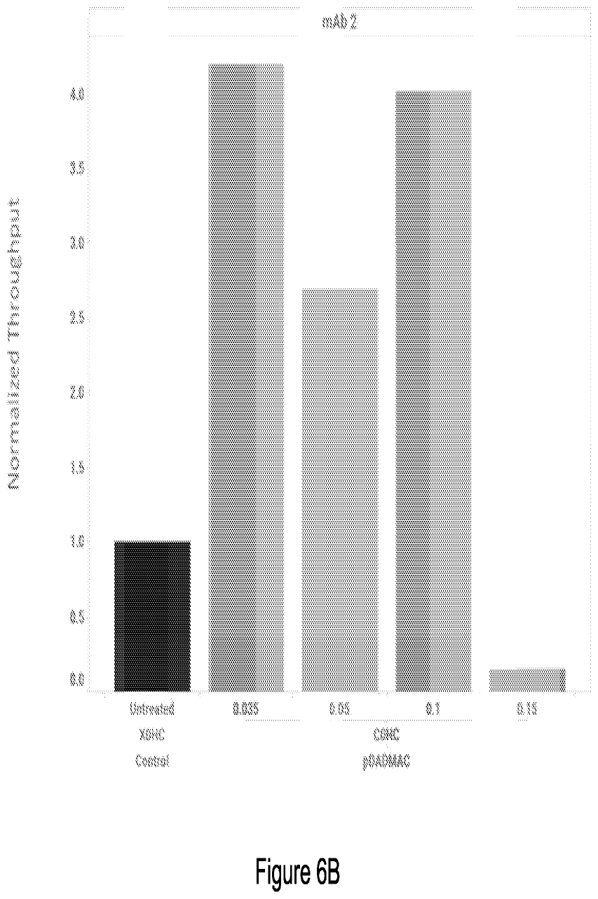

In harvest method of the disclosure, following single-use continuous solids discharge disc stack centrifugation and flocculation/precipitation, the centrate material is further purified using depth filtration. Accordingly, the fluid fraction of the centrate material following flocculation that was obtained from the large-scale harvests described in Example 1 were used for flocculation and small-scale depth filtration experiments to compare the depth filtration performance between untreated and pDADMAC-flocculated centrate, as briefly noted in Example 1, above. To 1 L centrate samples, pDADMAC (poly diallyldimethylammonium chloride, Sigma Aldrich, St. Louis, MO) stock solution was added to a final concentration of 0.025-0.01% (w/w) pDADMAC. A 1 L sample from each centrate was untreated to act as a treatment control. The pDADMAC solution was continuously added to the centrate for about one minute and was left to stir for at least 30 minutes, at 10° C. Stirring was achieved using a stir plate and stir bar; the stirring rate was varied to maintain constant mixing of the centrate with the flocculated material. A total of 56 conditions were tested across different modalities and different depth filters. Results showed that 0.035-0.10% (w/w) pDADMAC defined an optimal concentration range for flocculation of a variety of centrates containing target proteins (see FIG. 5 and FIG. 6). The data show optimal and sub-optimal but still effective flocculant concentrations, and consistent with that data, the disclosure contemplates the range of 0.025-0.15% flocculant for use in the harvest methods of the disclosure.

Filters with larger pore size distribution, such as C0HC and C0SP, may not be appropriate for untreated material, since the size distribution of particles in the untreated centrate of various molecules was measured to be in the range of 1 μm-60 μm using dynamic light scattering (DLS), with most particles in the range of 1-40 μm. This particle-size range coincides with the nominal pore size distributions of these filters. In addition, there is an increased number of finer particles in the untreated material, which can cause internal filter clogging and further exacerbate the depth filtration throughput. In contrast, pDADMAC flocculation works by aggregating finer molecules together into larger particles. DLS data showed that upon pDADMAC treatment, the number of particles was reduced, and the average particle size was increased. This may explain the increased throughput of depth filters used after pDADMAC flocculation; the larger particles are retained by the filter and prevent internal clogging compared to untreated material that contains a larger number of finer particles, constituting a higher fraction of the total particles being filtered. Selecting the correct treatment type, treatment concentration and depth filter nominal pore size rating are relevant to optimizing depth filter loading, step yield, and impurity removal.

Six small-scale micro-pod depth filters were tested, MILLISTAK+® X0HC, MILLISTAK+® X0SP, MILLISTAK+® C0HC, MILLISTAK+® C0SP (MilliporeSigma, Burlington, Mass), SARTOCLEAR® DL60, and SARTOCLEAR® DL75 (Sartorius, Bohemia, NY). The Millipore and Sartorius depth filters were 23 cm² and 25 cm², respectively.

The depth filters used for the small-scale experiments varied in pore-size distribution and material composition. The Millipore X0HC and C0HC depth filters are comprised of cellulose and an inorganic filter aid. The X0HC pore size ranged from 0.1-0.5 μm, the C0HC depth filter pore size ranged from 0.3-1.0 μm. Millipore X0SP and C0SP depth filters are synthetic with the same pore size distribution as the X0HC and C0HC depth filters. The Sartorius DL60 and DL75 have a wider range of pore size distribution compared to Millipore depth filters, with 0.6-10 μm and 2-14 μm nominal pore size distribution, respectively. The DL60 depth filter has a pure cellulose pre-filter combined with a coarse secondary filter. The DL75 depth filter contains a coarse primary grade filter with a secondary fine filtration grade filter. The variety of pore size distribution and material characteristics in depth filters used for the development ensured a large depth filtration data set was available to support the appropriate depth filter selection for the disclosed harvest methodology.

Prior to loading the treated and untreated samples, depth filters were flushed with a purified water or buffer solution at a temperature of about 10° C. and at a flow rate less than 400 liters per square meter per hour (LMH) flux to achieve a target throughput of at least 100 L/m². After the primary depth filter was flushed, a secondary 0.2 μm Optiscale Capsule SHC filter was attached to the primary filter (MilliporeSigma, Burlington, Mass) and this secondary filter was flushed at the same flux rate with target throughput of at least 50 L/m². The surface of the secondary filter was 3.6 cm², leading to a depth filtration surface ratio of stage 1 to stage 2 of 6.38 and 7.14 for Millipore and Sartorius depth filters, respectively.

After the water flush, the inlet of the primary depth filter was drained to remove excess water. The depth filters were then primed with their respective loads (untreated centrate or flocculated centrate) to ensure bubble removal. The filters were then loaded with either 1 L of untreated centrate or 1 L of the flocculated centrate. The inlet flux during the experiment was maintained between 90 LMH and 150 LMH. The final depth filtration throughput varied depending on the performance of the depth filtration. The turbidity of the depth filtrate pool was measured at the end of the experiment.

The depth filtration experiment ended when the stage 1 inlet pressure in the filter reached at least 20 psig or once the desired throughput was achieved. In some cases, the depth filtration experiment ended before the desired throughput was reached due to time constraints. Based on the data resulting from both depth filtration and flocculation studies, a pDADMAC range of 0.02-0.1% (w/w) was shown to be an optimal concentration range for inducing beneficial flocculation in the harvest methods of the disclosure.

The following parameters were included to assess the performance of depth filtration at small-scale: depth filtration final throughput, depth filtration yield, depth filtrate pool host cell proteins (HCP) levels (otherwise known as CHOP levels), depth filtrate pool DNA levels, normalized depth filtration throughput, HCP log reduction values (LRV) and DNA LRV. The normalized values were calculated using an untreated X0HC depth filter as a control condition and dividing the final values of various parameters by the control condition (X0HC+untreated). Normalized values show the magnitude increase or decrease in various parameters tested and thus help to better assess the degree of improvement of the new harvest technology. LRVs were calculated by applying a base ten logarithm transformation to the measured load value divided by the measured pool value.

Improved performance in depth filtration was characterized by an increased depth filtration throughput value or normalized depth filtration throughput value, increased or comparable percent yield, and increased or comparable HCP LRV and/or DNA LRV.

FIG. 1 shows the depth filtration normalized throughput values for various molecules, treatment types and depth filters. Normalized depth filtration magnitude for flocculated centrate conditions ranged from 0.85- to 16.2-fold compared to the magnitude of untreated centrate. Three main treatment types were tested: control (X0HC+untreated), pDADMAC-treated (various depth filters+0.05% (w/w) pDADMAC flocculation) and untreated (various depth filters other than X0HC+untreated). All conditions were run at less than or equal to 150 LMH flux. In general, the data show a much higher depth filtration throughput using 0.05% (w/w) pDADMAC flocculation compared to control conditions using a X0HC depth filter and untreated centrate, independent of the final % PCV of the material and the sample tested. Specifically, all depth filters performed better with 0.05% (w/w) pDADMAC flocculation except for mAb1 and X0HC. Control and untreated conditions reached maximum pressure of no more than 20 psig within a short period of time, whereas the pDADMAC-treated conditions were maintained with differential pressure of less than 10 psig and could have been operated for longer periods of time. This is advantageous because it indicates that higher volumes of material can be filtered before peak pressure is reached. Peak pressure of 20 psig was deemed a hazard and a safety risk, hence limiting depth filtration operation.

FIG. 2 shows that the percent yield for 0.05% (w/w) pDADMAC treatment conditions were comparable or higher than both control conditions and untreated conditions. The depth filters that were an exception were X0SP and X0HC paired with pDADMAC treatment. As a result, the use of X0SP or X0HC filtration with pDADMAC-treated centrates is not preferred due to lower yields. Control and untreated conditions showed lower yield, which is explained by depth filter clogging leading to maximum pressure being reached. In these experiments, no recovery buffer flush was performed.

The small-scale flocculation and depth-filtered pools shown in FIG. 1 were assessed for impurity levels. The depth filtrate pool impurity levels were measured and compared to the centrate pool using a log reduction value (LRV) calculation. High impurity clearance is observed when a high LRV value is observed. The HCP and DNA values for pDADMAC-treated (dark grey), untreated (light gray) and control conditions (black) are shown in FIG. 3 and FIG. 4. Variation in the impurity LRV was observed between centrate lots but in general the impurity LRV for pDADMAC-treated centrate was similar to or better than the untreated centrate and control conditions.

The disclosure provides a harvest method that takes advantage of single-use continuous solids discharge disc stack centrifugation in combination with flocculation of the centrate, e.g., using pDADMAC treatment for flocculation, and depth filtration that leads to a higher harvest yield than the traditional perfusion culture harvest methods using microfiltration. Molecules produced in continuous-perfusion culture, such as those disclosed in Example 1, have high PCVs in the range of 16% to 30%. High PCV cell cultures challenge existing microfiltration harvest technology, leading to variable harvest yields and high cost due to increased filter use. Additionally, some molecules experience low harvest yield (about 70%), resulting from product loss attributable to cell culture variability, filter variability and equipment variations collectively leading to varying amounts of product lost during the harvest operation. Current intermittent solids discharge centrifuge technology is not able to handle such high PCV cell culture harvests due to clogging of the centrifuge. It is recognized that continuous solids discharge disc stack centrifugation may not provide sufficient clarification of high solid content cell culture harvests and may not offer a robust harvest process option on its own or even in combination with depth filtration, as seen in the low throughput and in some cases low percent yield, HCP clearance and DNA clearance in the untreated centrate examples. It is the combination of continuous solids discharge disc stack centrifugation followed by flocculation that leads to robust processes for harvesting a wide variety of target proteins from high solid content perfusion cell cultures, and this process also renders feasible the incorporation of a depth filtration step to further purify a target protein while retaining high yield in a cost-effective series of steps, compatible with further conventional downstream purification operations.

Treating the centrate with 0.05% (w/w) pDADMAC led to aggregation of smaller particles, reducing the total amount of particles while effectively increasing the particle size distribution such that, when paired with an appropriate depth filter (e.g., Millistak+® C0HC, Millistak+® C0SP, Sartoclear® DL75, or Sartoclear® DL60), the centrifugation and flocculation steps led to increased depth filtration throughput, while providing similar or improved harvest yield, HCP clearance and DNA clearance when harvesting most target proteins. Additionally, pDADMAC flocculation was able to maintain low differential pressures throughout the run, which is an important aspect in terms of both safety and the ability to filter the desired amount of material while meeting target depth filtration throughput goals (see FIG. 8).

### Example 3 Effects of pDADMAC Concentrations on Turbidity and Small-Scale Depth Filtration Performance

pDADMAC dosing studies were performed to understand the effect of pDADMAC concentration on the turbidity of the flocculated samples. Dosing studies were repeated using centrate with variation in turbidity. Variation in centrate turbidity was generated by change the modality and the single-use continuous solids discharge centrifuge operating conditions as described in Table 2. Dosing studies were performed by adding pDADMAC from a stock solution to 40 mL of centrate to the final pDADMAC concentrations as shown in Table 2. The control (0% w/w pDADMAC) and the pDADMAC-treated centrate were mixed for at least 15 min. The samples were them centrifuged at 3000 rpm for 17 min using a JS4.2 centrifuge (Beckman Coulter, Indianapolis, IN). After centrifugation, the supernatant was placed in a new conical tube and the turbidity of the supernatant was measured using a Hach 2100P Portable Turbidimeter (Hach Company, Loveland, CO).

TABLE 2
pDADMAC dosing studies for mAb 1 and xmAb 1

| Single Use Continuous Centrifuge Parameters | | | | | | | | | | |
|---|---|---|---|---|---|---|---|---|---|---|
| | | Initial | | | | | | | | |
| | | centrate | Pump | Inlet | | | | | | |
| Modality | | turbidity | Speed | Flow Rate | pDADMAC Dosing Studies | | | | | |
| (Run) | Condition | (NTU) | (RPM) | (LPM) | pDADMAC concentration range (% w/w) | | | | | |
| mAb 1 | 1 | 547 | 5400 | 1.5 | 0 | 0.025 | 0.04 | 0.06 | 0.08 | 0.1 |
| (Run 2) | 2 | 740 | 6400 | 1.5 | 0 | 0.025 | 0.04 | 0.06 | 0.08 | 0.1 |
| | 3 | 663 | 6720 | 1.7 | 0 | 0.025 | 0.04 | 0.06 | 0.08 | 0.1 |
| | 4 | 740 | 5760 | 1.3 | 0 | 0.025 | 0.04 | 0.06 | 0.08 | 0.1 |
| xmAb 1 | 1 | 1500 | 6204 | 1.5 | 0 | 0.02 | 0.04 | 0.06 | 0.08 | 0.1 |
| (Run 1) | 2 | 250 | 6294 | 1.3 | 0 | 0.02 | 0.04 | 0.06 | 0.08 | 0.1 |

FIG. 5 shows the results of the pDADMAC dosing study as described in Table 2. FIG. 5A shows the results from the pDADMAC dosing study using mAb 1 (Run 2) from Table 1 and conditions described in Table 2. This study evaluated the impact of pDADMAC concentration on turbidity levels of various centrate sources subjected to different centrifuge flow rates and bowl speeds. Note that Table 1 only shows average values of the feed flow rate and bowl speed. Study results revealed low supernatant turbidity values in the pDADMAC concentration range of 0.04 to 0.10% (w/w). pDADMAC concentration levels lower than 0.04% were not desirable as this led to high turbidity levels.

Depicted in FIG. 5B are the results from the pDADMAC dosing study from xmAb1 (Run 1). This study evaluated the impact of pDADMAC concentration on centrate sources with different initial turbidity levels. The different initial turbidity in the centrate was generated by varying the inlet flow rate of the single use centrifuge. High turbidity and low turbidity centrate yielded initial turbidity values of 1500 NTU and 290 NTU, respectively. Study results showed low supernatant turbidity values in the pDADMAC concentration range of 0.02 to 0.10% (w/w), regardless of the initial centrate turbidity levels. pDADMAC concentration levels lower than 0.02% were not desirable as this did not reduce turbidity levels post pDADMAC treatment.

After determining the optimal pDADMAC concentration that led to the lowest turbidity values, small-scale depth filtration studies were performed using pDADMAC at concentrations of 0.02% (w/w), 0.05% (w/w), and 0.1% (w/w) to determine the concentration range that leads to the highest depth filtration throughput. The small-scale depth filter operation was executed as described in Example 2.

Monoclonal antibody mAb 1 (Run 1) and mAb 2 were harvested from cell culture broth using a single-use continuous solids discharge disc stack centrifuge, as described in Example 1, Table 1. pDADMAC flocculation was performed at small-scale using the 2% pDADMAC stock solution. For mAb 1 (Run 1), the pDADMAC concentration range tested was 0.01%, 0.02% and 0.05%. For mAb 2, the pDADMAC concentration range tested was 0.035%, 0.05%, 0.1% and 0.15%. All pDADMAC concentrations used in this experiment were (w/w). C0HC depth filters were used for pDADMAC treated samples. For each experiment, untreated centrate was filtered with a X0HC depth filter as a control.

Normalized depth filter throughput values from these experiments are shown in FIG. 6. Normalized depth filter throughput was calculated by dividing the small-scale depth filtration final throughputs (L/m²) of the pDADMAC-treated conditions by the final depth filtration throughput of the control untreated condition. Normalized throughput greater than one show improved depth filtration performance relative to the control. pDADMAC concentration between 0.035% and 0.1% (w/w) pDADMAC showed improved depth performance when compared to the control.

### Example 4 Effects of pDADMAC and PEG on Small-Scale Depth Filtration Performance

Centrate from mAb 1 Run 3 was collected. This large scale run is not included in Example 1 since it was not generate using single-use continuous solids discharge centrifuge equipment and instead was executed using a stainless steel continuous solids discharge centrifuge. The average centrate turbidity was 182 NTU which was similar to average centrate turbidity for mAb 1 Run 1 and Run 3, therefore it was deemed to be a representative material for pDADMAC treated depth filtration studies. The centrate obtained from the large scale was subjected to flocculation with 0.05% (w/w) pDADMAC alone or in combination with 3% PEG 3000, followed by depth filtration, as described in Example 2. pDADMAC and PEG 3000 solutions were simultaneously added to the centrate. The flocculated centrate was mixed for at least 30 minutes before loading onto a C0HC or C0SP depth filters. Untreated centrate was loaded onto an X0HC depth filter as a control because this had previously been determined to provide the highest depth filter load capacity for untreated centrate. The X0HC filter was used for the untreated control centrate because it is the best filter type for the size of particles in solution for the untreated condition (see FIG. 1). We used this as a control to understand how each molecule was similar/different from each other.

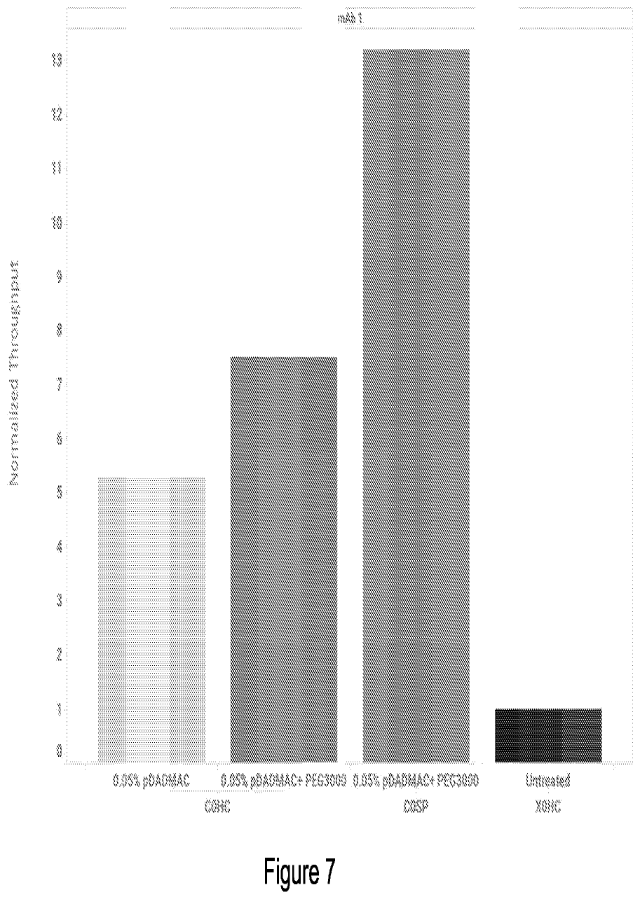

FIG. 7 shows that the pDADMAC+PEG3000 condition achieved increased depth filtration throughput compared to 0.05% w/w pDADMAC alone. Thus, the disclosure provides efficient and effective target protein harvest methods in which a continuous solids discharge disc stack centrifugation precedes a flocculation step that preferably uses 0.02%-0.15% pDADMAC with or without about 3% PEG, such as 3% PEG3000.

### Example 5 Comparison of Large-Scale and Small-Scale Depth Filtration Performance

mAb 1 Run 1, xmAb Run 1, xmAb 1 Run 2 and Bsp 2, as depicted in Table 1, were subjected to both large-scale and small-scale depth filtration. At large-scale, centrate was collected from single-use continuous solids discharge centrifugation as described in Table 1 for mAb 1 Run1, xmAb1 Run 1, xmAb1 Run 2 and Bsp 2. The centrate was subjected to flocculation and depth filtration at large-scale. Large-scale depth filtration was executed using C0HC depth filters. The filter size of C0HC used for large scale runs was greater than or equal to 1.1 m². A small-scale depth filtration study was performed in parallel for each centrate lot using untreated centrate and X0HC depth filters as a control. The depth filters for both small-scale and large-scale depth filters were flushed with DI water as described in Example 2. Flocculation was performed using a 2% (w/w) pDADMAC stock solution to achieve a final pDADMAC concentration of 0.05% (w/w) in the centrate. Each small-scale depth filtration condition was performed using at least 1 L of flocculated material. Small-scale depth filtration performance was assessed using a flocculated centrate depth filtration flow rate of 150 LMH for each of the three representative runs. Large-scale depth filtration performance for mAb 1 Run 1, xmAb1 Run 1, xmAb1 Run 2 and Bsp 2 were assessed using flocculated centrate flow rates of 65, 95, 155 LHM and 120 LMH, respectively.

FIG. 8 shows the depth filtration performance for large-scale flocculated centrate runs using a C0HC depth filter and 0.05% (w/w) pDADMAC treatment compared to small-scale depth filtration performance using X0HC depth filters. The data show that the flocculation and C0HC depth filtration performance (grey) is improved in terms of differential pressure trends when compared to control conditions using untreated centrate and X0HC depth filtration (black). pDADMAC treatment and C0HC filtration showed stable differential pressure below 10 psig. Maintaining a low differential pressure in manufacturing is a significant goal because a maximum allowable pressure during the process of about 20 psig is desired in order to maintain safety and reduce high pressure related risk. Ensuring that a low differential pressure can be maintained for a longer duration leads to more material being filtered, and this reduces any impact on downstream steps in the harvest operation. The harvest step yield for these large-scale runs is summarized in Table 3. Step yields ranged from 86% to 92% showing an improvement over the average microfiltration harvest step yield of approximately 70%.

TABLE 3
Summary of large-scale single-use continuous solids
discharge disc stack centrifuge, flocculation
and depth filtration process performance.

| | Average | Flocculation and | |
|---|---|---|---|
| | Centrifuge | Depth Filtration | Overall Harvest |
| Run ID | Yield (%) | Step Yield (%) | Yield (%) |
| mAb 1 (Run 1) | 93 | 96 | 89 |
| xmAb 1 (Run 1) | 93 | 95 | 88 |
| xmAb 1 (Run 2) | 90 | 96 | 86 |
| Bsp 2 | 94 | 98 | 92 |

### Example 6 Product Quality Considerations

Product quality was assessed by subjecting the depth filtrate pools containing target protein to preparative protein A purification followed by analytical testing by chromatographic fractionation or electrophoresis. Types of analytical chromatography used included size exclusion chromatography (SE), and cation exchange chromatography (CEX). Electrophoretic assessment of product quality was determined using reduced capillary electrophoresis sodium dodecyl sulfate (rCE), and non-reduced capillary electrophoresis (nrCE). These different types of analyses were conducted to provide a rigorous assessment of product quality.

FIG. 9 shows xmAb 1 Run 1 product quality comparisons of the pDADMAC-treated C0HC filtrate pool (dark grey), the untreated centrate C0HC pool (light grey) and the untreated X0HC filtrate pool (black). The C0HC condition was performed using 0.05% (w/w) pDADMAC as described in Example 2. As depicted in FIG. 9, the product quality assessments using SE, CEX, rCE, and nrCE, respectively, showed comparable results among the three types of materials being subjected to quality assessments, i.e., untreated centrate, pDADMAC-treated centrate and C0HC depth filtration pool. These results establish that treatment of cell culture centrifugation centrate with a flocculant such as pDADMAC does not negatively affect the product quality profile.

What is claimed is:

1. A method for separating a recombinant protein produced in a perfusion culture from at least one other perfusion culture component comprising:
(a) harvesting a pool or eluate stream from a perfusion culture comprising the recombinant protein and at least one other perfusion culture component, the perfusion culture having a packed cell volume (PCV) of at least about 16% at harvest;
(b) introducing the harvest pool or eluate stream into at least one continuous solids discharge disc stack centrifuge;
(b) operating the continuous solids discharge disc stack centrifuge, thereby separating fluid components into a centrate and a high-density composition;
(c) collecting the centrate;
(d) adding a flocculant to the centrate; and
(e) subjecting the centrate to a filtration step.

2. The method of claim 1, wherein the continuous solids discharge disc stack centrifuge is a single-use continuous solids discharge disc stack centrifuge.

3. The method of claim 1 or 2, wherein the flocculant is added to the centrate at a temperature of 8° C. to 12° C.

4. The method of claim 3, wherein the flocculant is added to the centrate at a temperature of about 10° C.

5. The method of any one of claims 1-4, wherein the perfusion culture is harvested from single-use bioreactor.

6. The method of claim 5, wherein the single-use bioreactor is at least 500 L.

7. The method of claim 5, where in the single-use bioreactor is 2,000 L or more.

8. The method of any one of claims 1-7, wherein the prefusion culture harvest pool or eluate stream had a turbidity of at least 180 NTU at harvest.

9. The method of any one of claims 1-8, wherein the perfusion culture harvest pool or eluate stream had a viable cell density of at least 2×10⁷ viable cells/ml at harvest.

10. The method of claim 9, wherein the perfusion culture harvest pool or eluate stream had a viable cell density of at least 3×10⁷ viable cells/ml at harvest.

11. The method of claim 9 wherein the perfusion culture harvest pool or eluate stream had a viable cell density of at least 5×10⁷ viable cells/ml at harvest.

12. The method of any one of claims 1-11, wherein the perfusion culture harvest pool or eluate stream had a packed cell volume of at least about 18% at harvest.

13. The method of claim 12, wherein the perfusion culture had a packed cell volume of at least about 20% at harvest.

14. The method of claim 12, wherein the perfusion culture had a packed cell volume of at least about 24% at harvest.

15. The method of claim 12, wherein the perfusion culture had a packed cell volume of at least about 26% at harvest.

16. The method of claim 12, wherein the perfusion culture had a packed volume of at least about 30% at harvest.

17. The method of any one of claims 1-16, wherein the temperature of the perfusion culture at harvest is about 8° C. to about 12° C.

18. The method of claim 17, wherein the temperature of the perfusion culture at harvest is about 10° C.

19. The method of any one of claims 1-18, wherein a continuous harvest eluate stream from the perfusion culture is introduced into the continuous solids discharge disc stack centrifuge.

20. The method of any one of claims 1-19, wherein a discontinuous batch of perfusion harvest pool is introduced into the continuous solids discharge disc stack centrifuge.

21. The method of any one of claims 1-20, wherein the turbidity of the of the centrate is greater than about 160 NTU.

22. The method of any one of claims 1-21, wherein the flocculant is poly(diallyldimethylammonium chloride) (pDADMAC).

23. The method of claim 22, wherein poly(diallyldimethylammonium chloride) (pDADMAC) as flocculant is added to at least 0.04% (w/w).

24. The method of claim 22, wherein poly(diallyldimethylammonium chloride) (pDADMAC) as flocculant is added to 0.04-0.15% (w/w).

25. The method of claim 22, wherein poly(diallyldimethylammonium chloride) (pDADMAC) as flocculant is added to 0.05% (w/w).

26. The method of any one of claims 1-25, wherein the recombinant protein is a eukaryotic protein.

27. The method of claim 26, wherein the eukaryotic protein is a mammalian protein.

28. The method of claim 27, wherein the mammalian protein is an antigen-binding protein.

29. The method of claim 28, wherein the antigen-binding protein is a monoclonal antibody, a bispecific antibody, a multispecific antibody, a bispecific T-cell engager molecule (BiTE®).

30. The method of any one of claims 1-29, wherein the protein is a granulocyte colony-stimulating factor, an erythropoiesis stimulating agent, a HER receptor, a cell adhesion molecule, a growth factor, an osteoinductive factor, insulin, a coagulation protein, a colony stimulating factor, a blood group antigen; a growth hormone, a growth hormone receptor, a T-cell receptor; a neurotrophic factor, a neurotrophin, a relaxin, an interferon, an interleukin, a viral antigen, a lipoprotein, an integrin, a rheumatoid factor, an immunotoxin, a surface-membrane protein, a transport protein, a homing receptor, an addressin, a regulatory protein, or an immunoadhesin.

31. The method of any one of claims 1-30, wherein the filtration includes a depth filter.

32. The method of claim 31, wherein the depth filter is MILLISTAK+® C0HC filter, a MILLISTAK+® C0SP filter, a SARTOCLEAR® DL60 filter, or a SARTOCLEAR® DL75 filter.

33. The method of claim 31, wherein the depth filter is a MILLISTAK+® C0HC filter or a MILLISTAK+® C0SP filter.

34. The method of any one of claims 31-33, wherein the centrate passes through the depth filter at a flux rate of 150 LMH or less and at a pressure of 10 psi or less.

35. The method of claim 34, wherein the pressure is 2 psi or less.

36. The method of claim 34, wherein the flux rate is 90 to 150 LMH.

37. The method of any one of claims 1-36, further comprising at least one additional chromatography step.

38. The method of claim 35, wherein the chromatography step is selected from ion exchange chromatography, hydrophobic interaction chromatography, or multimodal chromatography.

39. The method of any one of claims 1-38, further comprising at least one or more viral filtration step, viral inaction step, and/or UFDF step.

40. A target protein made by the method of any one of claims 1-39.

41. A method for maintaining low differential pressure during depth filtration of a load feed derived from a perfusion culture, the method comprising the steps:
(a) obtaining a flocculated centrate derived from a perfusion culture having packed cell volume (PCV) of at least 16% at the time of harvest;
(b) passing the centrate through a depth filter at a flux rate of 150 LMH or less and a pressure of 10 psi or less; and
(c) recovering the eluate.

42. A method for producing an isolated, purified recombinant protein from a perfusion culture initiated in a single use bioreactor comprising the steps of
a. inoculating the bioreactor with cells engineered to recombinantly express a protein of interest;
b. culturing the cells until the perfusion culture has a packed cell volume of at least 16%;
c. reducing the temperature of the culture to between 8° C. and 12° C. and collecting the perfusion culture from the bioreactor as a harvest pool or eluate stream;
d. introducing the harvest pool or eluate stream into at least one continuous solids discharge disc stack centrifuge, wherein the harvest pool or eluate stream is between 4° C. and 12° C.;
e. collecting a centrate from the centrifuge;
f. adding a flocculant to the centrate at a temperature of 8° C. and 12° C.;
g. passing the flocculated centrate through a depth filter at a at a flux rate of 90 to 150 LMH and at a pressure of 10 psi or less, wherein the flocculated centrate is between 4° C. and 12° C.;
h. subjecting the filtered centrate to one or more chromatography, filtration, and or UFDF unit operations; and
i. obtaining an isolated, purified, recombinant protein.

43. A pharmaceutical composition comprising the isolated, purified, recombinant protein produced with the method of claim 40.
